(19) United States
(12) Patent Application Publication
(10) Pub. No.: US 2015/0018223 A1
(43) Pub. Date: Jan. 15, 2015

(54) METHODS OF DIAGNOSING TAU-ASSOCIATED NEURODEGENERATIVE DISEASES

(71) Applicants: Garth F. Hall, Sudbury, MA (US); Wonhee Kim, Lowell, MA (US); Sangmook Lee, Westborough, MA (US)

(72) Inventors: Garth F. Hall, Sudbury, MA (US); Wonhee Kim, Lowell, MA (US); Sangmook Lee, Westborough, MA (US)

(73) Assignee: UNIVERSITY OF MASSACHUSETTS LOWELL, Lowell, MA (US)

(21) Appl. No.: 13/574,985

(22) PCT Filed: Jan. 28, 2011
(86) PCT No.: PCT/US2011/023065

§ 371 Date: Feb. 7, 2013

Related U.S. Application Data
(60) Provisional application No. 61/299,198, filed on Jan. 28, 2010.

(51) Int. Cl. G01N 33/68

(52) U.S. Cl. CPC G01N 33/6896; G01N 2333/4709; G01N 2800/2828; G01N 2800/2821; G01N 2800/2835
USPC 506/9; 436/501; 435/7.92; 436/86; 506/18

(57) ABSTRACT

This invention provides methods and kits for the detection of tau-associated neurodegenerative diseases, such as Alzheimer's disease, prior to the onset of clinical symptoms. The method generally involves determining the amount of one or more tau protein isoforms in a biological sample relative to a suitable control, where an altered amount of the tau isoform(s) relative to the control identifies the presence of a tau-associated neurodegenerative disease. Such methods can also be applied more generally for the detection of tau abnormalities in any biological sample.

### BACKGROUND

Neurodegenerative diseases are a major public health issue. For example, an estimated 4 million Americans currently have Alzheimer's disease (AD) and by the year 2030 it is predicted that approximately 1 in every 80 persons in the U.S. will suffer from the disease. Currently, neurodegenerative diseases can only be diagnosed when clinical symptoms present, which may be after irreversible neuronal damage has already occurred. This is a particular problem with AD, which usually cannot be diagnosed definitively even with the onset of clinical symptoms, which are usually first diagnosed more generally as mild cognitive impairment (MCI). Clinicopathological studies suggest that even MCI only becomes evident a number of years after the first pathological hallmarks of AD appear (Bennett et al. Neurology 2005 vol 5 pp 834-841). Accordingly, one of the major challenges in treating neurodegenerative diseases such as AD will be to diagnose individuals prospectively, i.e. prior to the emergence of clinical symptoms, in order to begin treatment before irreversible damage has set in.

There are currently few valid diagnostic biomarkers that can be used for early-stage diagnosis of any neurodegenerative disease. For AD, the most generally accepted current art biomarkers (i.e. high phosphotau/tau and tau/beta amyloid-42 ratios in the CSF) are not considered sufficiently sensitive and specific to be used as definitive AD diagnostics even after the onset of MCI, but have some use in identifying those MCI patients (perhaps half of the total) who will eventually progress to definitive AD. However, since the presence of MCI may already mark significant neuron loss in the brain, there is a need in the art for new methods of prospectively diagnosing neurodegenerative diseases as noted above.

### SUMMARY OF THE INVENTION

This invention provides methods and kits for the detection of tau-associated neurodegenerative diseases, such as Alzheimer's disease, prior to the onset of clinical symptoms. The method generally involves determining the amount of one or more tau protein isoforms in a biological sample relative to a suitable control (e.g., secreted tau), where an altered amount of the tau isoform(s) relative to the control identifies the presence of a tau-associated neurodegenerative disease. Such methods can also be applied more generally for the detection of tau abnormalities in any biological sample. As such the invention provides methods important in the diagnosis, for example, in the early diagnosis of tau associated neurodegenerative diseases. In essence, the invention provides novel biomarkers for identifying patients at risk for developing tau associated neurodegenerative disorders and particularly features detecting secreted tau as a biomarker for said diseases. The invention contemplates any means of detecting secreted tau as a biomarker. In preferred aspects, the invention features methods of detecting particular tau isoforms in, for example, CSF and/or methods of identifying tau as a component of the secretory apparatus of neuronal cells.

Accordingly, in one aspect the invention provides methods for identifying a subject having, or at risk of developing, a tau-associated neurodegenerative disease comprising:

obtaining a biological sample from the subject and determining the amount of tau 2+ protein in the biological sample relative to a suitable control, wherein a decreased amount of tau 2+ protein relative to the control identifies the subject as having, or being at risk of developing, a tau-associated neurodegenerative disease; and/or,

obtaining a biological sample from the subject and determining the amount of tau 2− protein in the biological sample relative to a suitable control, wherein a increased amount of tau 2− protein relative to the control identifies the subject as having, or being at risk of developing, a tau-associated neurodegenerative disease; and/or;

obtaining a biological sample from the subject and determining the tau 2+/tau 2− ratio in the biological sample relative to a suitable control, wherein a decreased tau 2+/tau 2− ratio relative to the control identifies the subject as having, or being at risk of developing, a tau-associated neurodegenerative disease.

The methods of the invention can be used to diagnose a tau-associated neurodegenerative disease in any animal subject. In certain embodiments, the subject is a mammal, e.g., a human. In certain embodiments, the subject has no clinically discernible cognitive impairment or is pre-symptomatic (i.e., displays no symptoms of neurodegenerative disease). In certain embodiments, the subject has no clinically discernable symptoms of a tau-associated neurodegenerative disease, for example, Alzheimer's disease. In certain embodiments, the subject has mild cognitive impairment (MCI) but has no clinically discernable symptoms of a tau-associated neurodegenerative disease, for example, Alzheimer's disease. In certain embodiments the subject has mild cognitive impairment, known in the art as the stage between normal forgetfulness and dementia.

Any tau-associated neurodegenerative disease can be diagnosed using the methods of the invention, including, without limitation Alzheimer's Disease, corticobasal degeneration, Pick's Disease, progressive supernuclear palsy, granulovacuolar disease, frontotemporal dementia, Lewy Body disease, Creutzfeld-Jacob Disease (CJD), variant Creutzfeld-Jacob Disease, and new variant Creutzfeld-Jacob Disease.

In another aspect, the invention provides methods for identifying a tau abnormality in a biological sample (e.g., as a biomarker of a tau associated disorder or disease), the methods comprising:

determining the amount of tau 2+ protein in the biological sample relative to a suitable control, wherein a decreased amount of tau 2+ protein relative to the control identifies a tau abnormality; and/or

determining the amount of tau 2− protein in the biological sample relative to a suitable control, wherein a increased amount of tau 2− protein relative to the control identifies a tau abnormality.

determining the tau 2+/tau 2− ratio in the biological sample relative to a suitable control, wherein a decreased tau 2+/tau 2− ratio relative to the control identifies a tau abnormality.

In another aspect, the invention provides methods for identifying tau as a component of secretory vesicles of neuronal cells (e.g., as a biomarker of tau associated disorders or diseases). Such methods comprise obtaining a population of neuronal cells; determining the amount of secreted vesicular tau, for example, tau 2+ relative to a suitable control, wherein an increased amount of tau as a secreted component of said neuronal cells identifies a tau abnormality.

The present invention also contemplates detecting secreted tau, for example, in CSF, followed by confirmatory screening featuring detection of secreted vesicular tau.

Any biological sample containing a tau abnormality can be assayed using the methods of the invention. In certain embodiments, the biological sample is an extracellular fluid (i.e., secreted tau is detected). Suitable extracellular fluids include, without limitation, cerebrospinal fluid, blood, serum and plasma.

In certain embodiments, the biological sample is a cell sample or media thereof or cell culture supernatant. Suitable cell samples, cell media samples or cell culture supernatant, without limitation include, for example, neuronal cell samples or brain cell samples.

In other embodiments, the biological sample is a tissue sample or tissue lysate sample. Suitable tissue samples or lysate samples, without limitation include, for example brain tissue samples, tissue samples from the spinal cord or any tissue samples from the nervous system.

In one embodiment the cell, cell media sample or cell culture supernatant or the tissue or tissue lysate sample has been fractioned to separate microvesicles, for example, exosomes.

In one embodiment, the instant invention features a two step method, wherein secreted tau (e.g., tau 2−, tau 2+ or a ratio of tau 2+/tau 2−) is detected in a sample (e.g., an extracellular fluid sample, e.g., CSF), the method further comprising detecting secreted tau (e.g., tau 2−, tau 2+ or a ratio of tau 2+/tau 2−) as a secretory component in a cell sample, e.g., a neuronal cell sample, for example, detection in a microsomal cell fraction, or in exosomes.

The methods of the invention include assaying the biological sample for tau secretion as a biomarker for tau associated disorders or diseases. In certain embodiments, tau secretion is assayed by detecting the tau isoforms in microvesicles. In certain embodiments, tau secretion is assayed by detecting tau isoforms in exosomes.

In one embodiment, tau secretion is assayed by determining whether tau is present in the media that hosts a cell sample or a tissue sample.

The methods of the invention can employ any art recognized means for the identification of tau 2+ protein, tau 2− protein or the tau 2+/tau 2− ratio. In certain embodiments, the amount of tau 2+ protein, tau 2− protein or the tau 2+/tau 2− ratio is determined by Western Blotting, ELISA, dot-blotting, high performance liquid chromatography (HPLC) and/or mass spectrometry. In a particular embodiment, tau 2+ protein is detected using an antibody that specifically binds to the amino acid sequence set forth in SEQ ID No.7. In a particular embodiment, tau 2+ protein is detected using an antibody that specifically binds to the amino acid sequence set forth in SEQ ID No.8. In a particular embodiment, tau 2− protein is detected using an antibody that specifically binds to the Exon 1/Exon 4 junction region of human tau. In a particular embodiment, tau 2− protein is detected using an antibody that specifically binds to the Exon 1/Exon 3 junction region of human tau.

The methods of the invention include assaying for tau secretion using art recognized methods, without limitation include, Western Blotting, ELISA, dot-blotting, high performance liquid chromatography (HPLC) and/or mass spectrometry. In a particular embodiment, the secreted tau resembles CSF tau species (e.g., cleavage fragments) associated with Alzheimer's disease. For example, tau secretion may be assayed by separating the cell media from the cells of the biological sample, assaying the cell media for the presence of tau, and comparing tau in the cell media sample to CSF tau species.

In another aspect, the invention provides kits for identifying a tau abnormality in a biological sample, the kit comprising one or more means for determining the amount of tau 2+ protein, tau 2− protein or the tau 2+/tau 2− ratio, and instructions for use of the kit to identify a tau abnormality in the sample. In certain embodiments the kit includes one or more antibodies (e.g., monoclonal antibodies, polyclonal antibodies, labeled and/or unlabeled) that specifically bind to tau 2+ protein and/or tau 2− protein. In a particular embodiment, the kit includes an antibody that specifically binds to the amino acid sequence set forth in SEQ ID No.7. In a particular embodiment, the kit includes an antibody that specifically binds to the amino acid sequence set forth in SEQ ID No.8. In a particular embodiment, the kit includes an antibody that specifically binds to the Exon1/Exon 4 junction region of human tau. In a particular embodiment, the kit includes an antibody that specifically binds to the Exon1/Exon 3 junction region of human tau.

In certain embodiments, the kit further comprises a means for obtaining the biological sample.

In certain embodiments, the kit further comprises a suitable control sample.

### BRIEF DESCRIPTION OF THE DRAWINGS

FIG. 1 depicts: A) a schematic diagram illustrating two different possible ways in which cleavage fragments of tau protein might appear in the CSF, i.e., via secretion from live cells into the extracellular space (left) or after neuronal death and autolysis (right); B) an Exon map showing the origin of the 6 tau isoforms expressed in human brain; and C) a diagram showing the tau splice variants tested in the Examples disclosed herein that are specifically secreted or retained by neurons (WT=wild type, mut=full length mutant, and frag=N terminal cleavage fragments).

FIG. 2 depicts an amino acid sequence alignment of the N-terminal region of all six human isoforms of tau.

FIG. 3 schematically depicts that tau binding to microtubules is normally controlled at specific sites on tau. Phosphorylation in the microtubule binding region (MTBR) (R1, R2, R3, R4) weakens tau binding to microtubules and favors tau aggregation. Phosphorylation of serine and threonine residues in the regions flanking the MTBRs reduce tau binding to microtubules and is associated with tau aggregation and toxicity.

FIG. 4 depicts the effects of tau alterations on cytotoxicity in neurodegenerative disease.

FIG. 5 schematically depicts that tau-induced degeneration is time and dose dependent and is accelerated by “tauopathy” mutations. A. graphically depicts that the ability to stage tau induced degeneration in ABCs makes it possible to show that this degeneration is progressive. B. graphically depicts that the rate of progression is increased with high levels of tau expressed (defined in relation to endogenous tubulin levels). C. schematically depicts that the presence of tauopathy mutations (G272V, P301L, V337M, R406W) increases the proportion of high stage cells seen at both early (10-20 days of expression) and late (30+ days of expression) times after plasmid injection relative to that seen with the expression of the parent WT isoform.

FIG. 6 depicts Western blots of cell lysates and culture media from cells expressing: A) endogenous tau; B) endogenous tubulin; C) exogenous 255 amino acid N-terminal fragments of tau in which, lacking or containing exons 2 and 3; and D) exogenous full length tau lacking or containing exons 2, or exons 2 and 3.

FIG. 7 depicts that tau secretion from mammalian neuroblastoma (NB2A) cells also requires the tau N-terminal domain. An N-terminal monoclonal antibody (tau 12, residues 9-18) recognizes secreted 1-255 and full length tau in concentrated culture medium conditioned by tau-expressing NB2A cells (asterisk). Secreted full-length tau species shows some C-terminal cleavage (caret). The bottom half of the figure demonstrates that when the blot was reprobed with tau 5 (specific for 210-235), the c-terminal (211-441) domain is retained in the lysate and is not secreted (asterisks). As depicted I the immunoblot, the tau 5 epitope is absent from the secreted tau. Tubulin immunolabel is retained in the lysate, which indicates that tau secretion is not merely an artifact of non-specific release from degenerating NB2A cells.

FIG. 8 depicts that tau secretion is inhibited by the presence of an N-terminal insert (exon 2) in MC1 and NB2A cells. A. This was observed for cells transfected (or induced) with both the N-terminal tau fragment (1-255) and Full length tau. B. depicts that E2− tau is secreted 10-15 times more efficiently than E2+ tau.

FIG. 9 depicts that the presence of tau microtubule binding regions (MTBR) affects whether tau secretion occurs focally or in a diffuse pattern. The immunoblot illustrates that secreted tau in culture occurs as two groups of bands that correspond to whether the microtubule binding region (MTBR) is present (top band—focal) or absent (bottom bands—diffuse).

FIG. 10, A. schematically depicts the tau protein. B. depicts that both diffuse and focal pathways of tau secretion from ABCs require the tau N terminal domain. C, depicts that overexpression of full length tau isoforms results in focal and diffuse tau secretion. D. depicts that deletion of the C terminal (MTBR) half of tau causes profuse secretion and abolishes “focal” deposits. E. depicts that deletion of the N-terminal half of tau blocks secretion of tau.

FIG. 11 depicts that tau secretion from lamprey ABCs is also inhibited by the presence of the N-terminal insert E2 in ABCs; A) Anterior Bulbar cells (ABCs) in the lamprey brain imaged live with GFP fluorescence (left) or fixed and immunolabeled with tau (red) and tubulin (green) (right); B) Transverse section through a lamprey brain immunostained with the N-terminal specific tau mAb, tau12; C) ABCs expressing 255 amino acid N-terminal tau fragments lacking or containing exons 2 and 3; and D) lamprey brain fixed and immunolabeled for secreted tau species using the phosphotau specific mAb AT180 (arrow, right) and total tau antibody (left). Scale Bars: 100 microns.

FIG. 12 depicts the use of monoclonal antibodies specific for E2− (secreted) and E2+(retained) tau. Monoclonal antibody 9A1 binds an epitope in the junction between exon 1 and exon 4, and is used to identify E2− (secreted) tau, and monoclonal antibody DC39E2 binds exon 2, and is used to identify E2+(retained) tau.

FIG. 13 depicts the expected results of a study comparing the sensitivity of the methods of the invention with conventional CSF assays. A and B show the experimental design and expected result of the proposed comparison between “total” CSF tau (All or T12+ tau), tau fragments containing the 2/3 insert (tau 2+ protein, or DC39N1+ tau) and tau phosphorylated at the AT180 or AT270 sites (P), which currently represent the most widely used measure of “phosphorylated tau.” C and D illustrate the use of an Exon1/Exon4 junction region specific antibody (9A1) to directly identify tau protein fragments lacking the exon 2/3 insert (tau 2− protein or “secreted tau”) and the expected tau 2+/tau 2− ratios.

FIG. 14, A. schematically illustrates that secreted tau is an early maker of tau-associated neurodegenerative disease, for example, Alzheimer's disease. A significant amount of secreted tau was observed in the CSF from pre-symptomatic subjects. A strong amount of secreted tau was observed in the CSF from subjects with mild cognitive impairment (MCI); B. schematically depicts the development of Alzheimer's disease as the illness proceeds from 1. The presymptomatic stage, 2. The preclinical stage, 3. Mild cognitive impairment (MCI) and finally to 4. Alzheimer's disease (AD).

FIG. 15 depicts that secreted tau resembles cerebrospinal fluid (CSF) tau species (cleavage fragments) associated with Alzheimer's disease. A. schematically depicts that CSF tau in AD is cleaved by disease activated enzymes (caspases and calpains) that degrade proteins at specific sites. B. depicts that secreted tau occurs primarily in two fragment sizes that resemble the caspase and calpain fragments seen in AD.

FIG. 16 depicts that tau associated with exosomes resembles CSF tau from early AD patients. B. depicts secreted “large fragment” tau that is phosphorylated at the AT270 site and is selectively enriched in exosomes.

FIG. 17 depicts the purification and characteristics of microvesicle and exosomal secreted tau. A. schematically depicts the purification of microvesicle secreted tau and the purification of exosomal secreted tau from media and CSF. The biological sample was subjected to several hard spins to remove large organelles from the sample. The sample was then spun in sucrose to fractionate the sample so that the fraction containing exosomes could be separated from the fraction containing other membranes, including microvesicles. B. depicts that secreted tau is enriched in exosome fractions of conditioned media samples from M1C cultures induced to overexpress E2− tau as determined by mass spectrometric analysis. The enrichment of exosomes for tau varies with tau phosphorylation state, with some phosphoepitopes (AT100 and PHF1) more prominent in secreted tau than in lysate exosome fractions and others (AT270, AT180) less so. This figures also depicts that all Alzheimer's disease associated phosphorylation site are found in exosomal tau.

FIG. 18 depicts that secreted tau is associated with vesicular elements positive for exosomal markers and AD-associated proteins. A. depicts that tau fractions contain 80 nm vesicles positive for the exosome marker Alix; Immunoelectron microscopy (IEM) of an M1C exosome fraction showing colloidal gold decoration of the widely accepted exosome marker protein alix on 100 nm vesicles (arrows) B. depicts that the proteins that co-purify with tau in exosomes are “classically” found in exosomes and/or associated with tau misprocessing in AD; tau was co-enriched with 4 classes of proteins in M1C exosomes. The largest of these was intrinsic or membrane associated proteins with signal transduction or vesicle trafficking functions. Such proteins are typically exosome-associated and included some (e.g. annexin 7, alix) that are considered exosome “markers”). Other co-enriched proteins included known tau binding proteins (fyn kinase, HSC70 associated protein) and AD associated proteins such as APP.

### DETAILED DESCRIPTION

This invention provides methods and kits for the detection of tau abnormalities in a biological sample. The invention is based on the surprising finding that tau secretion can serve as a biomarker for predicting or early-stage diagnosing of tau associated disorders. The invention is based, in part, on the discovery that human tau isoforms lacking the amino acids encoded by Exon 2 (herein tau 2− proteins) are secreted into the extracellular space in the brain, in a similar pattern to that seen in tau-associated neurodegenerative diseases, such as Alzheimer's disease. The methods of the invention generally involve determining the amount of tau 2+ protein or tau 2− protein, or the tau 2+/tau 2− ratio in a biological sample relative to a suitable control, where a decreased amount of tau 2+ protein, an increased amount of tau 2− protein, or a decreased tau 2+/tau 2− ratio relative to the control identifies the presence of a tau abnormality. Such methods and kits are particularly useful for the early detection of tau-associated neurodegenerative diseases, such as Alzheimer's disease, prior to the onset of clinical symptoms.

The invention is further based, at least in part, on the surprising discovery that tau isoforms, in particular, the tau 2− isoform is extracellularly secreted, for example, into the CSF by classical secretory mechanisms. In particular, tau is detectable in microvesicles, or exosomes, as a step leading to secretion into, for example, CSF. The invention is further based on the surprising discovery that tau secretion is a disease associated event that is a better and more useful indicator of future tau-associated neurodegenerative disease, for example, Alzheimer's disease, development than current markers of neuron death.

In order that the present invention may be better understood, certain terms are first defined.

As used herein, the term “tau” refers to the tau family of microtubule-associated proteins (Weingarten et al., 1975, PNAS. (72) 1858-1862), including, without limitation, the six known isoforms of human tau protein, the amino acid sequence of which have been assigned the following Genbank accession numbers: GI:82534351; GI:6754638; GI:8400711; GI:8400715; GI:178557736; and GI:178557734.

As used herein, the term “tau 2+ protein” refers to tau protein isoforms comprising the amino acids encoded by exon 2 of the human tau gene, or the functional equivalent of human exon 2 in other organisms.

As used herein, the term “tau 2− protein” refers to tau protein isoforms lacking the amino acids encoded by exon 2 and exon 3 of the human tau gene, or the functional equivalent of human exon 2 and exon 3 in other organisms.

As used herein, the term “tau 2+/tau 2− ratio” refers to the ratio of amount of tau 2+ protein to tau 2− protein in a sample.

As used herein, the term “tau-associated neurodegenerative disease” refers to any neurodegenerative disease in which tau production, phosphorylation, aggregation or accumulation by neurons and/or glia in the CNS is abnormal. Suitable diseases include, without limitation, Alzheimer's Disease, corticobasal degeneration, Pick's Disease, progressive supernuclear palsy, granulovacuolar disease, frontotemporal dementia, Lewy Body disease, Creutzfeld-Jacob Disease (CJD), variant Creutzfeld-Jacob Disease, and new variant Creutzfeld-Jacob Disease.

As used herein, the term “tau abnormality” refers to an abnormal amount of tau 2+ protein or tau 2− protein, or an abnormal tau 2+/tau 2− ratio in a biological sample relative to a suitable control.

As used herein, the term “amount”, with respect to tau 2+ protein or tau 2− protein, refers to either (a) an absolute amount of tau 2+ protein or tau 2− protein as measured in molecules, moles or weight per unit volume or (b) a relative amount of tau 2+ protein or tau 2− protein (e.g., measured by densitometric analysis). The amount of tau 2+ protein or tau 2− protein can be measured directly using standard protein assays (e.g., ELISA, mass spectrometry, and the like), or can be inferred from measuring the absolute or relative level of mRNA encoding tau 2+ protein or tau 2− protein in a sample.

As used herein, the term “suitable control” refers to any sample or reference value useful for identifying a tau abnormality in a biological sample. Suitable control samples include, without limitation, samples containing an amount of tau 2+ protein, or tau 2− protein (or possessing a tau 2+/tau 2− ratio) representative of that found in a normal subject (i.e., a subject in which tau secretion from neuronal cells is normal) and/or a subject known to have a tau-associated neurodegenerative disease (e.g., AD). Suitable reference values include, without limitation, the average amount of tau 2+ protein or tau 2− protein (or the tau 2+/tau 2− ratio) obtained from a population of normal subjects and/or a population of subjects known to have a tau-associated neurodegenerative disease (e.g., AD).

As used herein, the term “Exon 1/Exon 4 junction region” refers to any epitope in the amino acid sequence encoded by the junction of exon 1 and exon 4 of human tau that is specific to tau 2− proteins.

As used herein, the term “Exon 1/Exon 3 junction region” refers to any epitope in the amino acid sequence encoded by the junction of exon 1 and exon 3 of human tau that is specific to tau 2− proteins.

As used herein the term “exosome” refers to a small, e.g. about 50 to about 90 nm vesicle secreted by mammalian cells. Exosomes contain a wide variety of soluble proteins including, for example, proteins whose secretion correlates with various pathological states. An exosome is created intracellularly when a segment of the cell membrane invaginates and is endocytosed. This internalized segment is ultimately broken down into smaller vesicles that are subsequently expelled from the cell. Exosomes are secreted by cells under both normal and pathological conditions and, in the latter instance, present attractive candidates as detectable biomarkers.

As used herein, the term “specifically binds to” with reference to an antibody means that the antibody exhibits appreciable affinity for a particular antigen or epitope and, generally, does not exhibit significant crossreactivity. In exemplary embodiments, the antibody exhibits no crossreactivity (e.g., an antibody that specifically binds to tau2+ protein does not crossreact with tau2− protein and vice versa). “Appreciable” or preferred binding includes binding with an affinity of at least 10⁶, 10⁷, 10⁸, 10⁹ M⁻¹, or 10¹⁰ M⁻¹.

Multiple approaches may be used to “model” Alzheimer's Disease and related tauopathies. For example, cell culture models, transgenic models and/or in situ cellular models (e.g. lamprey giant neurons) may be used to model AD.

### I. Tau Proteins

Tau proteins are microtubule-associated proteins abundant in neurons of the central nervous system (Weingarten et al., 1975, supra). Tau proteins interact with tubulin through microtubule binding regions (MTBRs), for example, MTBRs R1, R2, R3 and R4, to stabilize microtubules and promote tubulin assembly into microtubules. Tau is phosphorylated in vivo by a host of kinases. Phosphorylation of serine and threonine residues in and flanking the MTBRs of tau weakens tau binding to microtubules, and is associated with tau aggregation and toxicity.

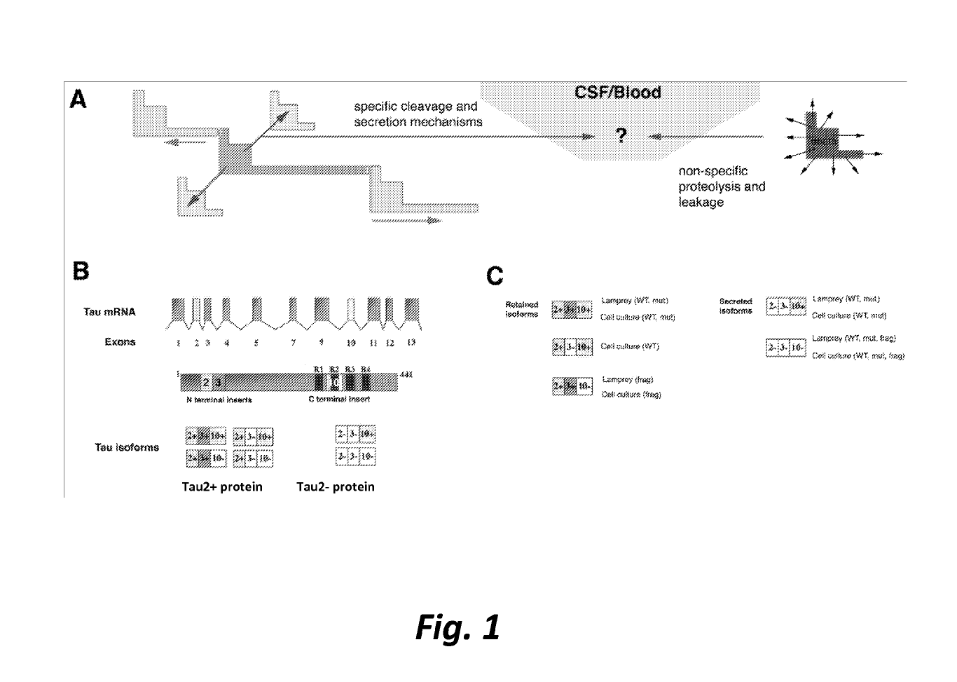

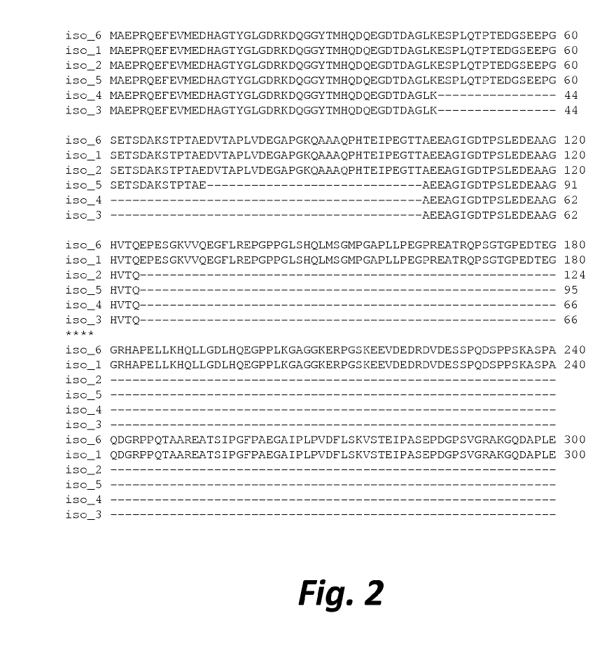

Hyperphosphorylation of the tau protein (tau inclusions), however, can result in the self-assembly of tangles of paired helical filaments and straight filaments, which are involved in the pathogenesis of Alzheimer's disease and other Tauopathies (Alonso et al., 2001, PNAS. (98) 6923-8). Furthermore, phosphorylation of N-terminal tyrosines occurs when tau interacts with the plasma membrane and functions in the secretion and interneuronal transfer of tau. Phosphorylation of all of these sites occurs in tau-associated neurodegenerative disease, for example, Alzheimer's disease. In humans, there are six known tau isoforms generated as a result of alternative splicing in exons 2, 3, and 10 of the tau gene (Groedert et al., 1989, Neuron, 3, 519-526; FIGS. 1 and 2). The amino acid sequences of the human tau isoforms are as follows:

Isoform 1

(SEQ ID NO. 1)

MAEPRQEFEVMEDHAGTYGLGDRKDQGGYTMHQDQEGDTDAGLKESPLQT
PTEDGSEEPGSETSDAKSTPTAEDVTAPLVDEGAPGKQAAAQPHTEIPEG
TTAEEAGIGDTPSLEDEAAGHVTQEPESGKVVQEGFLREPGPPGLSHQLM
SGMPGAPLLPEGPREATRQPSGTGPEDTEGGRHAPELLKHQLLGDLHQEG
PPLKGAGGKERPGSKEEVDEDRDVDESSPQDSPPSKASPAQDGRPPQTAA
REATSIPGFPAEGAIPLPVDFLSKVSTEIPASEPDGPSVGRAKGQDAPLE
FTFHVEITPNVQKEQAHSEEHLGRAAFPGAPGEGPEARGPSLGEDTKEAD
LPEPSEKQPAAAPRGKPVSRVPQLKARMVSKSKDGTGSDDKKAKTSTRSS
AKTLKNRPCLSPKHPTPGSSDPLIQPSSPAVCPEPPSSPKHVSSVTSRTG
SSGAKEMKLKGADGKTKIATPRGAAPPGQKGQANATRIPAKTPPAPKTPP
SSGEPPKSGDRSGYSSPGSPGTPGSRSRTPSLPTPPTREPKKVAVVRTPP
KSPSSAKSRLQTAPVPMPDLKNVKSKIGSTENLKHQPGGGKVQIINKKLD
LSNVQSKCGSKDNIKHVPGGGSVQIVYKPVDLSKVTSKCGSLGNIHHKPG
GGQVEVKSEKLDFKDRVQSKIGSLDNITHVPGGGNKKIETHKLTFRENAK
AKTDHGAEIVYKSPVVSGDTSPRHLSNVSSTGSIDMVDSPQLATLADEVS
ASLAKQGL
isoform 2

(SEQ ID NO. 1)

MAEPRQEFEVMEDHAGTYGLGDRKDQGGYTMHQDQEGDTDAGLKESPLQT
PTEDGSEEPGSETSDAKSTPTAEDVTAPLVDEGAPGKQAAAQPHTEIPEG
TTAEEAGIGDTPSLEDEAAGHVTQARMVSKSKDGTGSDDKKAKGADGKTK
IATPRGAAPPGQKGQANATRIPAKTPPAPKTPPSSGEPPKSGDRSGYSSP
GSPGTPGSRSRTPSLPTPPTREPKKVAVVRTPPKSPSSAKSRLQTAPVPM
PDLKNVKSKIGSTENLKHQPGGGKVQIINKKLDLSNVQSKCGSKDNIKHV
PGGGSVQIVYKPVDLSKVTSKCGSLGNIHHKPGGGQVEVKSEKLDFKDRV
QSKIGSLDNITHVPGGGNKKIETHKLTFRENAKAKTDHGAEIVYKSPVVS
GDTSPRHLSNVSSTGSIDMVDSPQLATLADEVSASLAKQGL
isoform 3

(SEQ ID NO. 1)

MAEPRQEFEVMEDHAGTYGLGDRKDQGGYTMHQDQEGDTDAGLKAEEAGI
GDTPSLEDEAAGHVTQARMVSKSKDGTGSDDKKAKGADGKTKIATPRGAA
PPGQKGQANATRIPAKTPPAPKTPPSSGEPPKSGDRSGYSSPGSPGTPGS
RSRTPSLPTPPTREPKKVAVVRTPPKSPSSAKSRLQTAPVPMPDLKNVKS
KIGSTENLKHQPGGGKVQIINKKLDLSNVQSKCGSKDNIKHVPGGGSVQI
VYKPVDLSKVTSKCGSLGNIHHKPGGGQVEVKSEKLDFKDRVQSKIGSLD
NITHVPGGGNKKIETHKLTFRENAKAKTDHGAEIVYKSPVVSGDTSPRHL
SNVSSTGSIDMVDSPQLATLADEVSASLAKQGL
isoform 4

(SEQ ID NO. 1)

MAEPRQEFEVMEDHAGTYGLGDRKDQGGYTMHQDQEGDTDAGLKAEEAGI
GDTPSLEDEAAGHVTQARMVSKSKDGTGSDDKKAKGADGKTKIATPRGAA
PPGQKGQANATRIPAKTPPAPKTPPSSGEPPKSGDRSGYSSPGSPGTPGS
RSRTPSLPTPPTREPKKVAVVRTPPKSPSSAKSRLQTAPVPMPDLKNVKS
KIGSTENLKHQPGGGKVQIVYKPVDLSKVTSKCGSLGNIHHKPGGGQVEV
KSEKLDFKDRVQSKIGSLDNITHVPGGGNKKIETHKLTFRENAKAKTDHG
AEIVYKSPVVSGDTSPRHLSNVSSTGSIDMVDSPQLATLADEVSASLAKQ
GL
isoform 5

(SEQ ID NO. 1)

MAEPRQEFEVMEDHAGTYGLGDRKDQGGYTMHQDQEGDTDAGLKESPLQT
PTEDGSEEPGSETSDAKSTPTAEAEEAGIGDTPSLEDEAAGHVTQARMVS
KSKDGTGSDDKKAKGADGKTKIATPRGAAPPGQKGQANATRIPAKTPPAP
KTPPSSGEPPKSGDRSGYSSPGSPGTPGSRSRTPSLPTPPTREPKKVAVV
RTPPKSPSSAKSRLQTAPVPMPDLKNVKSKIGSTENLKHQPGGGKVQIIN
KKLDLSNVQSKCGSKDNIKHVPGGGSVQIVYKPVDLSKVTSKCGSLGNIH
HKPGGGQVEVKSEKLDFKDRVQSKIGSLDNITHVPGGGNKKIETHKLTFR
ENAKAKTDHGAEIVYKSPVVSGDTSPRHLSNVSSTGSIDMVDSPQLATLA
DEVSASLAKQGL
isoform 6

(SEQ ID NO. 1)

MAEPRQEFEVMEDHAGTYGLGDRKDQGGYTMHQDQEGDTDAGLKESPLQT
PTEDGSEEPGSETSDAKSTPTAEDVTAPLVDEGAPGKQAAAQPHTEIPEG
TTAEEAGIGDTPSLEDEAAGHVTQEPESGKVVQEGFLREPGPPGLSHQLM
SGMPGAPLLPEGPREATRQPSGTGPEDTEGGRHAPELLKHQLLGDLHQEG
PPLKGAGGKERPGSKEEVDEDRDVDESSPQDSPPSKASPAQDGRPPQTAA
REATSIPGFPAEGAIPLPVDFLSKVSTEIPASEPDGPSVGRAKGQDAPLE
FTFHVEITPNVQKEQAHSEEHLGRAAFPGAPGEGPEARGPSLGEDTKEAD
LPEPSEKQPAAAPRGKPVSRVPQLKARMVSKSKDGTGSDDKKAKTSTRSS
AKTLKNRPCLSPKHPTPGSSDPLIQPSSPAVCPEPPSSPKHVSSVTSRTG
SSGAKEMKLKGADGKTKIATPRGAAPPGQKGQANATRIPAKTPPAPKTPP
SSATKQVQRRPPPAGPRSERGEPPKSGDRSGYSSPGSPGTPGSRSRTPSL
PTPPTREPKKVAVVRTPPKSPSSAKSRLQTAPVPMPDLKNVKSKIGSTEN
LKHQPGGGKVQIINKKLDLSNVQSKCGSKDNIKHVPGGGSVQIVYKPVDL
SKVTSKCGSLGNIHHKPGGGQVEVKSEKLDFKDRVQSKIGSLDNITHVPG
GGNKKIETHKLTFRENAKAKTDHGAEIVYKSPVVSGDTSPRHLSNVSSTG
SIDMVDSPQLATLADEVSASLAKQGL

The invention is based, in part, on the discovery that human tau isoforms lacking the amino acids encoded by Exon 2 (herein tau 2− proteins) are secreted into the extracellular space in the brain, in a similar pattern to that seen in tau-associated neurodegenerative diseases, such as Alzheimer's disease. In the methods of the invention, the presence of abnormal amounts of tau 2+ protein, tau 2− protein or an abnormal tau 2+/tau 2− ratio in a sample the amounts of tau 2+ protein or tau 2− protein are used as an indicator of tau abnormalities and for the identification of a subject having, or at risk of developing, a tau-associated neurodegenerative disease.

Certain aspects of the invention are based on the surprising discovery that tau is an alternatively spliced protein and abnormal tau cleavage occurs in tau-associated neurodegenerative disease, for example, Alzheimer's disease. In certain embodiments, tau secretion is inhibited by the presence of an N terminal-insert (exon 2 and/or exon 3). In a preferred embodiment, tau secretion is inhibited by the presence of the exon 2 insert.

Detection reagents (e.g., antibodies) that specifically bind the amino acid sequence encoded by human exon 2 can be used to identify tau 2+ protein. For example, in certain embodiments of the invention, tau carrying the insert can be detected using monoclonal antibody DC39E2 which binds to exon 2.

Moreover, a subset of tau 2+ protein also contain exon 3, and thus, detection reagents (e.g., antibodies) that specifically bind to the amino acid sequence encoded by human exon 3 can also be used to identify tau 2+ protein. Conversely, detection reagents (e.g., antibodies) that specifically bind to epitopes present in the amino acid sequence encoded by the junction of human exon 1 and exon 4 or the junction of human exon 1 and exon 3, but not present in tau 2+ proteins, can be used to specifically identify tau 2− protein. For example, in certain embodiments of the invention, tau lacking the insert can be identified using monoclonal antibody 9A1, which binds to epitiopes present in the junction between exon 1 and exon 4.

The amino acid sequence encoded by human Exon 2 is as follows:

(SEQ ID No. 7)

ESPLQTPTEDGSEEPGSETSDAKSTPTAED.

The amino acid sequence encoded by human Exon 3 is as follows:

(SEQ ID No. 8)

DVTAPLVDEGAPGKQAAAQPHTEIPEGTT.

Phosphorylation of serine and threonine residues in, and flanking, the MTBRs of tau weakens tau binding to microtubules, and is associated with tau aggregation and toxicity. Hyperphosphorylation of the tau protein (tau inclusions), however, can result in the self-assembly of tangles of paired helical filaments and straight filaments, which are involved in the pathogenesis of Alzheimer's disease and other Tauopathies (Alonso et al., 2001, PNAS. (98) 6923-8). Furthermore, mutations that cause familial taupathies, for example, G272V, P301L and V337M, also cause tau hyperphosphorylation.

Moreover, phosphorylation of N-terminal tyrosines occurs when tau interacts with the plasma membrane and functions in the secretion and interneuronal transfer of tau.

Phosphorylation of these N-terminal and C-terminal phpsphorylation sites occurs in tau-associated neurodegenerative disease, for example, Alzheimer's disease.

In one aspect, the invention provides methods for assaying the basic cellular and intracellular events that give rise to neuronal death and dementia in tau-associated neurodegenerative disease, for example, Alzheimer's disease by analyzing the tau protein. In one embodiment of the invention, the fate and modifications of the pool of tau that is not bound to microtubules, but is associated with toxicity and degeneration is analyzed.

### II. Tau-Associated Neurodegenerative Diseases

In one aspect, the invention provides methods for identifying a subject having, or at risk of developing, a tau-associated neurodegenerative disease. Any neurodegenerative disease that exhibits an abnormal amount of tau 2+ protein or tau 2− protein, or an abnormal tau 2+/tau 2− ratio in the extracellular space in the brain will return a positive result using the methods of the invention. Such diseases include, without limitation, Alzheimer's Disease, corticobasal degeneration, frontotemporal lobar degeneration (also known as Pick's Disease), progressive supernuclear palsy, granulovacuolar disease, frontotemporal dementia, Lewy Body disease, Creutzfeld-Jacob Disease (CJD), variant Creutzfeld-Jacob Disease, and new variant Creutzfeld-Jacob Disease. Tau-associated neurodegenerative diseases of non-human animals, including, without limitation, mad cow disease or scrapie, can also be diagnosed by the methods of the invention using the appropriate test reagents.

The methods of the invention are especially useful for diagnosing individuals with AD. In general, AD is not inherited by a patient, but develops due to the complex interplay of a variety of genetic factors and, therefore, is extremely difficult to diagnose. Individuals suffering either sporadic or familial forms of AD are usually diagnosed following presentation of one or more characteristic symptoms of AD. Common symptoms of AD include cognitive deficits that affect the performance of routine skills or tasks, problems with language, disorientation to time or place, poor or decreased judgment, impairments in abstract thought, loss of motor control, mood or behavior alteration, personality change, or loss of initiative. The number deficits or the degree of the cognitive deficit displayed by the patient usually reflects the extent to which the disease has progressed. For example, the patient may exhibit only a mild cognitive impairment, such that the patient exhibits problems with memory (e.g. contextual memory) but is otherwise able to function well.

Several tests have been developed to assess cognitive skills or performance in human subjects, for example, subjects at risk for or having symptoms or pathology of dementia disorders (e.g., AD). Cognitive deficits can be identified by impaired performance of these tests, and many treatments have been proposed based on their ability to improve performance in these tests. Although some tasks have evaluated behaviors or motor function of subjects, most tasks have been designed to test learning or memory.

Cognition in humans may be assessed using a wide variety of tests including, but not limited to, the following tests. The ADAS-Cog (Alzheimer Disease Assessment Scale-Cognitive) is an 11-part test that takes 30 minutes to complete. The ADAS-Cog is a preferred brief exam for the study of language and memory skills See Rosen et al. (1984) Am J Psychiatry. 141(11):1356-64; Ihl et al. (2000) Neuropsychobiol. 41(2):102-7; and Weyer et al. (1997) Int Psychogeriatr. 9(2):123-38.

The Blessed Test is another quick (˜10 minute) test of cognition which assesses activities of daily living and memory, concentration and orientation. See Blessed et al. (1968) Br J Psychiat 114(512):797-811.

The Cambridge Neuropsychological Test Automated Battery (CANTAB) is used for the assessment of cognitive deficits in humans with neurodegenerative diseases or brain damage. It consists of thirteen interrelated computerized tests of memory, attention, and executive function, and is administered via a touch sensitive screen from a personal computer. The tests are language and largely culture free, and have shown to be highly sensitive in the early detection and routine screening of Alzheimer's disease. See Swainson et al. (2001) Dement Geriatr Cogn Disord.; 12:265-280; and Fray and Robbins (1996) Neurotoxicol Teratol.18(4):499-504. Robbins et al. (1994) Dementia 5(5):266-81.

The Consortium to Establish a Registry for Alzheimer's Disease (CERAD) Clinical and Neuropsychological Tests include a verbal fluency test, Boston Naming Test, Mini Mental State Exam (MMSE), ten-item word recall, constructional praxis, and delayed recall of praxis items. The test typically takes 20-30 minutes and is convenient and effective at assessing and tracking cognitive decline. See Morris et al. (1988) Psychopharmacol Bull. 24(4):641-52; Morris et al. (1989) Neurology 39(9):1159-65; and Welsh et al. (1991) Arch Neurol. 48(3):278-81.

The Mini Mental State Exam (MMSE) developed in 1975 by Folestein et al, is a brief test of mental status and cognition function. It does not measure other mental phenomena and is therefore not a substitute for a full mental status examination. It is useful in screening for dementia and its scoring system is helpful in following progress over time. The Mini-Mental State Examination MMSE is widely used, with norms adjusted for age and education. It can be used to screen for cognitive impairment, to estimate the severity of cognitive impairment at a given point in time, to follow the course of cognitive changes in an individual over time, and to document an individual's response to treatment. Cognitive assessment of subjects may require formal neuropsychologic testing, with follow-up testing separated by nine months or more (in humans). See Folstein et al. (1975) J Psychiatr Res. 12:196-198; Cockrell and Folstein (1988) Psychopharm Bull. 24(4):689-692; and Crum et al. (1993) J. Am. Med. Association 18:2386-2391.

The Seven-Minute Screen is a screening tool to help identify patients who should be evaluated for Alzheimer's disease. The screening tool is highly sensitive to the early signs of AD, using a series of questions to assess different types of intellectual functionality. The test consists of 4 sets of questions that focus on orientation, memory, visuospatial skills and expressive language. It can distinguish between cognitive changes due to the normal aging process and cognitive deficits due to dementia. See Solomon and Pendlebury (1998) Fam Med. 30(4):265-71, Solomon et al. (1998) Arch Neurol. 55(3):349-55.

In addition, a number of diagnostic tests are available for identifying individuals who have AD. These include measurement of CSF tau and Ab42 levels. Elevated tau and decreased Ab42 levels signify the presence of AD.

### III. Alzheimer's Disease

Alzheimer's disease (AD) is the most common form of dementia. AD is an incurable, degenerative and terminal disease. The two hallmarks of Alzheimer's Disease (AD) are the senile plaques and neurofibrillary tangles that spread through the brain as the disease develops and cause progressive dementia.

Neurofibrillary tangles (NFTs) are fibrillar intracellular aggregates composed mainly of abnormally polarized, phosphorylated and truncated tau protein.

Senile Plaques (SPs) are fibrillar deposits composed predominately of beta-amyloid peptide that devlope outside neurons in AD.

Abnormal, toxic interactions between tau protein and beta amyloid are the central factor driving neurodegeneration in AD. They may also play a critical role in how AD lesions spread within the brain. Furthermore, during AD pathogenesis abnormalities in tau occur ‘downstream” of amyloid beta.

The basic cellular and intercellular events that give rise to neuronal death and dementia in AD have been analyzed by studying the tau protein. It is known, for example, that a pool of tau that is not bound to microtubules is associated with toxicity and degeneration. Accordingly, studies have been performed to analyze the fate and modifications of tau.

The binding of tau to microtubules (MT) is normally controlled by phosphorylation at specific sites on tau. The phosphorylation of N terminal tyrosines may play a role in the secretion and interneuronal transfer of tau. For example, phosphorylation at 9G3 occurs when tau interacts with the plasma membrane.

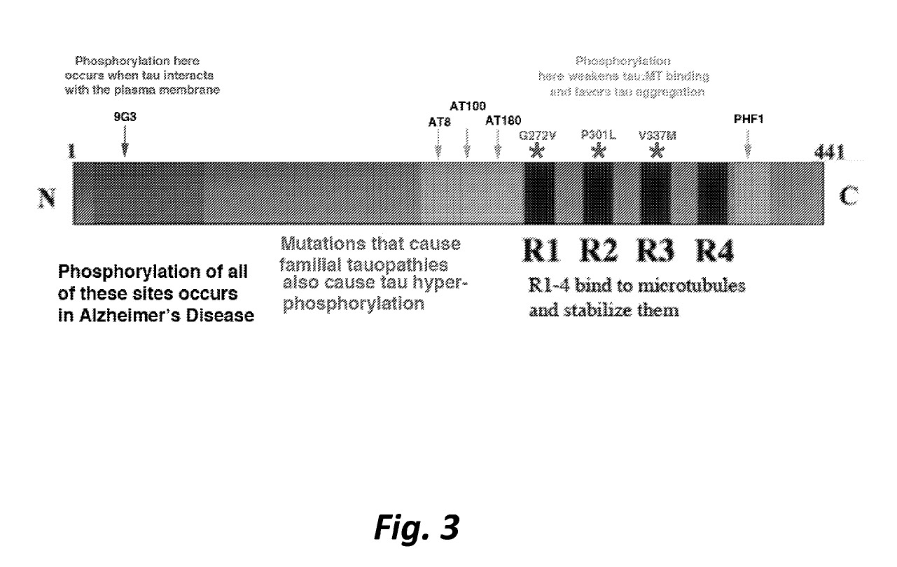

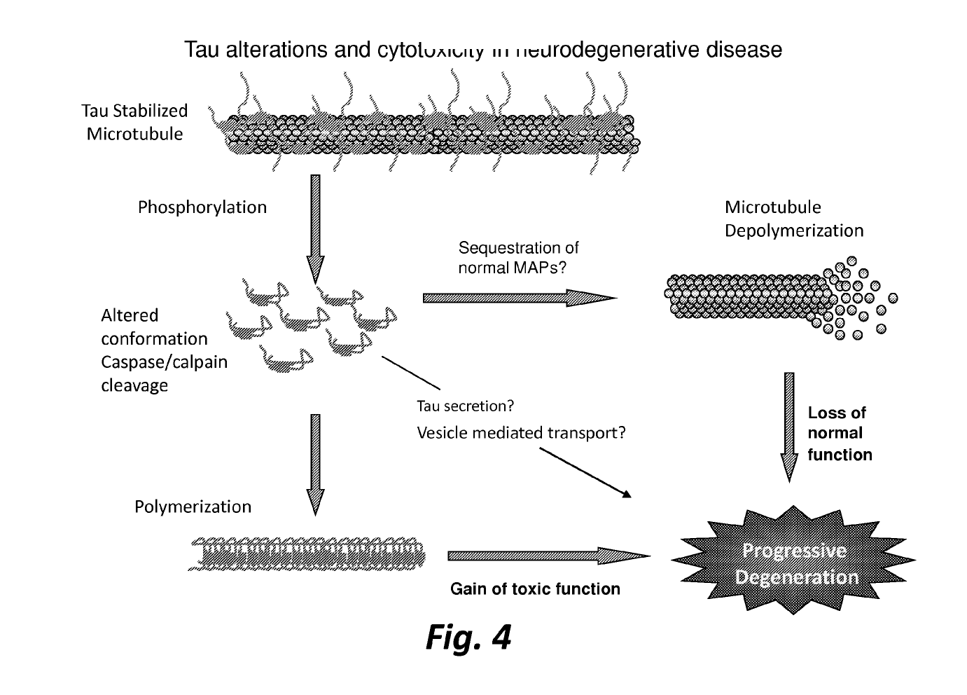

Microtubule binding regions (MTBRs) are located near the C-terminus of the tau protein and include the following regions: R1, R2, R3 and R4. Each of these regions (R1-4) bind microtubules and stabilize them. The instant invention provides that phosphorylation of Ser/Thr residues in the regions flanking the microtubule binding regions (MTBRs) reduce tau binding to MTs and is associated with tau aggregation and toxicity. Thus, phosphorylation in the MTBRs weakens tau-MT binding and favors tau aggregation. The phosphorylation sites in and around the MTBRs include AT8, AT100, AT180, G272V, P301L, V37M and PHF1. Furthermore, phosphorylation at least at one or more of these sites (e.g., 9G3, AT8, AT100, AT180, G272V, P301L, V37M and PHF1) occurs in subjects with Alzheimer's disease (FIGS. 3 and 4)

### IV. Detecting Tau Abnormalities

In certain embodiments, the methods of the invention are employed to detect the presence of abnormal amounts of tau 2+ protein or tau 2− protein, or an abnormal tau 2+/tau 2− ratio in a sample. In certain embodiments, the methods of the invention are employed to detect secreted tau, for example, the tau 2− protein, in subjects who are pre-symptomatic for tau-associated neurodegenerative disease, for example, Alzheimer's disease. In certain embodiments, the methods of the invention are employed to detect secreted tau, for example, the tau 2− protein, in subjects who are preclinical for tau-associated neurodegenerative disease, for example, Alzheimer's disease. In certain embodiments, the methods of the invention are employed to detect secreted tau, for example, the tau 2− protein, in subjects with mild-cognitive impairment or with mild Alzheimer's disease.

A sample may include, without limitation, blood serum or blood plasma, CSF, urine, cell culture supernatant and other liquid samples of biological origin from an individual or a set of individuals.

In certain embodiments, methods of the invention are employed to detect tau in the secretory apparatus of a cell, e.g., a neuronal cell. For example, tau secretion as evidenced by detection of tau in an exosome or microsomal fraction of a cell, e.g., a neuronal cell, can be used as a biomarker for tau associated disorders or diseases.

Samples may have been manipulated in any way after their procurement, for example, by electrical, chemical and/or mechanical treatments. For example, the sample may be treated with reagents, solublization, or enriched for certain components, such as proteins or peptides.

The tau 2+ protein or tau 2− protein can be detected by any suitable method including, without limitation, immunological-based methods, optical methods, fluorescent detection, spectrometric detection, chemiluminescent detection, matrix assisted laser desorption-time-of flight (MALDI-TOF) detection, high pressure liquid chromatographic detection (HPLC), charge detection, mass detection, radio frequency detection, or light diffraction detection.

In certain embodiments, any of a variety of known immunoassay methods can be used for detection and quantification of the tau 2+ protein or tau 2− protein including, but not limited to: immunoassay (e.g., by enzyme-linked immunosorbent assay (ELISA)) using an antibody that specifically binds to the tau 2+ protein (e.g., an antibody that specifically binds to the amino acid sequence set forth in SEQ ID No.7) or tau 2− protein (e.g., an antibody that specifically binds to the Exon1/Exon 4 junction region of human tau); and functional assays for tau 2+ protein or tau 2− protein, e.g., microtubule binding activity.

Immunofluorescence assays can be easily performed on CSF using a labeled antibody. It is also possible to perform such assays in blood, serum or blood plasma, urine if sufficient tau 2+ protein or tau 2− protein diffuses from human CSF to the plasma.

To increase the sensitivity of the assay, the immunocomplex (bound antibody and sample) may be further exposed to a second antibody (e.g., a reporter antibody), which is labeled and binds to the first antibody or to the biomarker. Typically, the secondary antibody comprises a detectable moiety, e.g., with a fluorescent marker so it can be easily visualized by any method (e.g., by eye, microscope, or machine).

In a particular embodiment, a sandwich ELISA or modified ELISA is used. In general, such methods comprise contacting the sample with an antibody that specifically binds to the tau 2+ protein or tau 2− protein. The antibody utilized may be any antibody, such as for example, monoclonal antibodies immobilized to a support. After allowing the sample time to bind with the antibody and washing of unbound sample, a labeled antibody is contacted with the sample or, in various embodiments, the capture antibody and sufficient time is allowed for the labeled antibody to specifically bind to the tau 2+ protein or tau 2− protein or the capture antibody. The bound label is detected and thus the tau 2+ protein or tau 2− protein is detected and can be quantified.

In certain embodiments, the tau 2+ protein or tau 2− protein is detected by mass spectrometry, or methods that employs a mass spectrometer to detect gas phase ions. Mass spectrometry methods are particularly useful for use in the methods of the invention because they allow for the simultaneous detection of tau 2+ and tau 2− proteins. Hence the tau 2+/tau 2− ratio can be easily determined in a single assay. Examples of mass spectrometers are time-of-flight, magnetic sector, quadrupole filter, ion trap, ion cyclotron resonance, electrostatic sector analyzer and hybrids of these. In such embodiments, the relative levels of tau 2+ protein or tau 2− protein in a sample can be determined with mass spectrometry where a standard curve can be generated using corresponding synthetic peptides without isotope labeling. Alternatively, the tau 2+ protein or tau 2− protein in the sample can be identified and quantified when the identical synthetic peptides are isotope labeled and spiked in the sample.

The mass spectrometer may be a laser desorption/ionization mass spectrometer. In laser desorption/ionization mass spectrometry, the analytes are placed on the surface of a mass spectrometry probe, a device adapted to engage a probe interface of the mass spectrometer and to present an analyte to ionizing energy for ionization and introduction into a mass spectrometer. A laser desorption mass spectrometer employs laser energy, typically from an ultraviolet laser, but also from an infrared laser, to desorb analytes from a surface, to volatilize and ionize them and make them available to the ion optics of the mass spectrometer.

In general, the sample obtained from an individual is contacted with an adsorbent surface for a period of time sufficient to allow tau 2+ protein or tau 2− protein present in the sample to bind to the adsorbent surface. After an incubation period, the substrate is washed to remove unbound material. Any suitable washing solutions can be used; such as an aqueous solution. The extent to which molecules remain bound can be manipulated by adjusting the stringency of the wash. The elution characteristics of a wash solution can depend, for example, on pH, ionic strength, hydrophobicity, degree of chaotropism, detergent strength, and temperature. An energy absorbing molecule is then applied to the substrate and the bound tau 2+ protein or tau 2− protein is then detected in a gas phase ion spectrometer such as a time-of-flight mass spectrometer or an ion trap mass spectrometer. The tau 2+ protein or tau 2− protein is ionized by an ionization source such as a laser, the generated ions are collected by an ion optic assembly, and then a mass analyzer disperses and analyzes the passing ions. The detector then translates information of the detected ions into mass-to-charge ratios. Detection of tau 2+ protein or tau 2− protein may involve the detection of the signal intensity. Thus, both the quantity and mass of the tau 2+ protein or tau 2− protein can be determined.

In another mass spectrometry method, tau 2+ protein or tau 2− protein may be first captured on a chromatographic resin that binds it. For example, the resin can be derivatized with an antibody. Alternatively, this method could be preceded by chromatographic fractionation before application to the bio-affinity resin. After elution from the resin, the sample can be analyzed by MALDI, electrospray, or another ionization method for mass spectrometry. In another alternative, one could fractionate on an anion exchange resin and detect by MALDI or electrospray mass spectrometry directly. In yet another method, one could capture the tau 2+ protein or tau 2− protein on an immuno-chromatographic resin that comprises antibodies that bind the tau 2+ protein or tau 2− protein, wash the resin to remove unbound material, elute the bound molecules from the resin and detect the eluted proteins by MALDI, electrospray mass spectrometry or another ionization mass spectrometry method.

In certain embodiments, detection of tau 2+ protein or tau 2− protein can be accomplished using capture reagents that specifically bind to the tau 2+ protein or tau 2− protein (e.g., an antibody that specifically binds to the amino acid sequence set forth in SEQ ID No.7 or the Exon1/Exon 4 junction region of human tau). In general, the capture reagent may be bound (e.g., covalently or non-covalently, via hydrophobic or hydrophilic interactions, H bonding, or van der Waals etc.) to a solid phase, such as a bead, a plate, a membrane or a chip. Methods of coupling biomolecules, such as antibodies or antigens, to a solid phase are well known in the art. They can employ, for example, bifunctional linking agents, or the solid phase can be derivatized with a reactive group, such as an epoxide or an imidizole, that will bind the molecule on contact.

In certain embodiments, biochips may be employed. The surfaces of biochips may be derivatized with the capture reagents directed against the tau 2+ protein or tau 2− protein. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound thereto. Thus, addressable arrays can be created to capture, detect and quantify one or more biomarkers in addition to the tau 2+ protein or tau 2− protein.

Exosomes can be purified using any one of a variety of art-recognized methods. In general, exosomes are purified from lysed cell samples by differential centrifugation and require, for example, sucrose gradient centrifugation to achieve purity and homogeneity of the purified exosome preparations. Exemplary techniques can be found, for example, in “Isolation and Characterization of Exosomes from Cell Culture Supernatants and Biological Fluids”, Théry et al., Current Protocols in Cell Biology 2006 April; Chapter 3: Unit 3.22.

The skilled artisan will appreciate that to assay for tau abnormalities in a non-human subject, the amino acid sequence of the region of tau analogous to human exon 2 or the human exon1-4 junction region in that non-human subject can be used as readouts for tau 2+ proteins and tau 2− proteins respectively. Such analogous regions can be easily identified by, for example, sequence similarity algorithms (e.g., BLAST) or manual inspection of the amino acid sequence.

The methods of the invention can be used either alone or in concert with any one or more art recognized assays for employed for the diagnosis of tau-associated neurodegenerative diseases.

### V. Exosome Association of Secreted Tau Proteins

The instant inventors have demonstrated that tau protein co-purifies with exosomes, which are a marker of a clearly characterized, yet unconventional mode of secretion from many cell types, including neurons. The proteins that co-purify with tau in exosomes are mostly membrane associated proteins that are either known exosome markers such as alix and annexin 7 or are from protein families that have been shown to be in exosomes. Other proteins that are co-enriched with tau in exosomes (and which themselves are known to be exosome associated) have art-recognized links to tau processing in AD. For example, fyn kinase is known to play a role in disease-associated tau misprocessing that could plausibly account for the localization of tau to exosomes in the early stages of AD and/or other tauopathies. Interestingly, exosomal tau purified from the media of neuronal cells in culture is somewhat dephosphorylated relative to most CSF tau, strengthening the idea that it is derived from a different source than the tau found in established AD cases, which is presumably due to release after neuron death. Exosomes are present in high concentration in human CSF, along with tau and other neurodegenerative disease markers, but no one has yet attempted to associate any of these markers selectively with exosomes. The findings of the instant inventors a) provide a biological mechanism that confirms the validity of tau secretion, and b) greatly increases the validity of tau secretion as a disease-associated event that can serve as a better and more useful indicator of future AD development than current art markers of neuron death (e.g., phosphorylated tau).

The prior art does not indicate that tau is exosome associated. Furthermore, there has been a widespread assumption in the art that tau is cytosolic, is not secreted and that the tau commonly found in the CSF of AD patients is exclusively due to the passive release of tau from dead neurons. However, three other proteins that drive neurodegeneration in AD and other neurodegenerative diseases (i.e. alpha synuclein, beta amyloid and prion protein), often in association with tau, have been shown to be present in exosomes.

Based at least in part on the above, the invention features the novel finding that tau isoforms can be used as biomarkers for early diagnosis of Alzheimer's as certain forms of tau are secreted in CSF. The instant invention also features the novel finding that tau isoforms are secreted in exosomes.

In a preferred embodiment of the invention AD is diagnosed using a two step process. The first step comprises determining if Tau isoforms are present in the CSF, and the second step comprises purifying the exosomal particles from, e.g., neuronal cells or CSF and identifying the Tau isoforms.

### VI. Kits

In another aspect, the invention provides kits for identifying a tau abnormality in a biological sample, the kit comprising: a) means for determining the amount of the tau 2+ protein or tau 2− protein, or the tau 2+/tau 2− ratio; and b) instructions for use of the kit to identify a tau abnormality in the sample.

Suitable means for determining the amount of the tau 2+ protein or tau 2− protein, or the tau 2+/tau 2− ratio include, without limitation, the assays and reagents discussed supra. In certain embodiments the kit includes one or more antibodies (e.g., monoclonal antibodies, polyclonal antibodies, labeled and/or unlabeled) that specifically bind to tau 2+ protein and/or tau 2− protein. In a particular embodiment, a supplied antibody specifically binds to the amino acid sequence set forth in SEQ ID No.7. In another particular embodiment, a supplied antibody specifically binds to the amino acid sequence set forth in SEQ ID No.8. In another particular embodiment, the supplied antibody specifically binds to the Exon1/Exon 4 junction region of human tau. In another particular embodiment, the supplied antibody specifically binds to the Exon1/Exon 3 junction region of human tau.

The kit may optionally provide additional components that are useful in the procedure, including, but not limited to, buffers, developing reagents, labels, reacting surfaces, control samples, and standards.

The kit may further provide a means for isolating the biological sample.

### Example 1

In this Example, suitable materials and methodologies for carrying out the subsequent Examples are described.

### Plasmids

Plasmids used in this study were derived from pRc/CMVn123c and pRcCMVn1234, as described in Arai et al. 2004, Ann. Neurol. 55, 72-79. Both plasmids lack two N-terminal insert regions and contain three or four microtubule binding repeat sequences respectively. GFP (green fluorescence protein) is fused to N-terminus of T23 (no N-terminal insert and three C-terminal repeats) and T24 (no N-terminal insert and four C-terminal repeats). N-terminal and C-terminal constructs express 1-255 and 211-441 amino acids respectively of T23. The GFP/T23 bicistronic construct expresses GFP from the SV40 promoter and T23 from the CMV promoter separately.

### Cell Culture and Transfection

Transfection of N2BA cells was carried out using Lipofectamine™ 2000 (Sigma Aldrich) according to the manufacturer's protocol. Serum-free medium was replaced with complete medium 24 hours after the transfection. Successfully transfected cells were localized by GFP fluorescence. Transfection rates under this condition routinely exceeded 70%. Culture medium was collected 24 hours after medium replacement and cleared by centrifugation at 10,000×g at 4° C. for 10 minutes to remove cells and cellular debris. To concentrate protein, Centricon (Millipore, Billerica, Mass.) was used according to the manufacturer's protocol. NB2a/d1 cells were lysed in Tris-NaCl (TN) buffer (50 mM Tris-HCl, pH 7.4, 150 mM NaCl, 1% Triton-X, 10% Glycerol, 2 mM EDTA, protease inhibitor cocktail (Sigma protease inhibitors). Cell lysates were clarified by centrifugation at 10,000×g for 10 minutes.

### Immunoblotting

Cell lysates and medium were analyzed by Western blot. After running the gels, samples were transferred to the polyvinyledene difluoride (PVDF) membrane and incubated with the following primary antibodies: Tau12 (1:10,000), Tau5 (1:10,000), and DM1A (1:1,000). Horseradish peroxidase conjugated anti-rabbit (1:10,000) and anti-mouse (1:10,000) antibodies were applied as second antibodies. Western images were obtained using a chemiluminescence (Pierce, Rockford, Ill.).

Surgery and Plasmid Microinjection into ABCs

Plasmid microinjection was performed as previously described in Lee et. al 2009. J. Alz Dis vol 16, pp 99-111, 2008. Neurobiology of Aging 30, 34-40. Briefly, an anesthetized lamprey hindbrain was exposed and the identified anterior bulbar cells were injected with the plasmid with 0.5% fast green at a final concentration of lmg/ml. A total of approximately 1100 lampreys were used for in situ microinjections, with an expression rate of between 1-2 anterior bulbar cells per lamprey injected. A total of 460 tau-expressing cells were identified for use in this study.

### Immunohistochemistry

Immunohistochemistry for brightfield microscopy was done as previously described in Lee et. al., supra. Briefly, lamprey brains expressing GFP tagged human tau expressing cells were identified under the fluorescence and fixed in FAA (10% formalin, 10% glacial acetic acid, and 80% ethanol). Immunohistochemistry was performed on 10 μm transverse sections of paraffin-embedded lamprey heads. Sections containing somatodendritic regions or axons were stained with following monoclonal tau antibodies. Tau5 and Tau12 (1:1000; a generous gift from L. Binder, Northwestern University, Chicago, Ill.) were preliminary used to identify the tau expressing cells in lamprey brain. For immunolabeling tyrosine phosphorylation of tau at residue 18, monoclonal 9G3 (1:100) was used. Polyclonal anti-GFP (1:400; Invitrogen, Carlsbad, Calif.) was used to detect GFP itself or tagged proteins. Appropriate species specific VECTASTAIN ABC kits (Vector Laboratories, Burlingame, Calif.) were used and diaminobenzidine (Sigma Aldrich) was used as a chromagen.

For confocal imaging of multiple immunoprobes, immunofluorescence was used. Deparaffinized and dehydrated sections were placed in pre-heated Tris-EDTA pH 9.0 buffer (10 mM Tris Base, 1 mM EDTA Solution, 0.05% Tween 20, pH 9.0) for unmasking the antigens and epitopes in brain tissue sections. The sections were then incubated with 0.2% triton in TBS for 20 minutes for permeabilization. For quenching autofluorescence, 2 mg/ml sodium borohydride in TBS was applied to the sections for 10 minutes twice. Each section was blocked with 5% goat serum with 0.1% fish gelatin in TBS for 1 hour at room temperature. After blocking, the sections were incubated overnight at 4° C. with two or three of the following primary antibodies: Tau5 (1:400), Tau12 (1:400), 9G3 (1:30), and anti-GFP (1:200). For confocal immunofluorescence imaging, species-specific or mouse IgG subclass specific secondary antibodies linked to FITC, Rhodamine Red-X, cy5 (1:200; Jackson ImmunoResearch Laboratories, Inc., West Grove, Pa.) were used. Confocal imaging was obtained using a Fluoview 300 (FV300) Confocal Laser Scanning Microscope (Olympus, Center Valley, Pa.). Three dimensional image files were collected, created and analyzed with NIH Image J (v. 1.41×) and Volocity 4.3 (Improvision, Waltham, Mass.).

### Example 2

This Example illustrates that tau induces progressive degeneration in lamprey anterior bulbar cells (ABCs) that resembles the neurodegeneration seen in AD. Lamprey ABCs possess the ideal combination of accessibility, large size and stereotyped morphology for use in an in situ cellular model. Normal ABC morphology features large and ellipsoidal somata, axons which are straight unbranched processes that extend caudally and ipsilaterally from the soma along the midline in the ventral tracts for most of the length of the spinal cord and dendrites, which are divided into two major domains, one extending laterally from the soma and the other medially. The lateral dendritic domain consists of 1 or 2 thick profusely branched primary dendrites which extend from the lateral aspect of the soma for approximately 150 m before turning ventromedially. The medial domain is smaller and more variable, comprising one or a few primary dendrites extending ventromedially from the soma for 50-100 m.

Interestingly, ABCs expressing human tau undergo progressive tau-specific dendritic degeneration. Dendritic degeneration occurs over several weeks following expression of exogenous tau via plasmid injection, beginning with the distal most dendrites (e.g., at about 17 days) and proceeding proximally with time (e.g., at about 28 days).

### Example 3

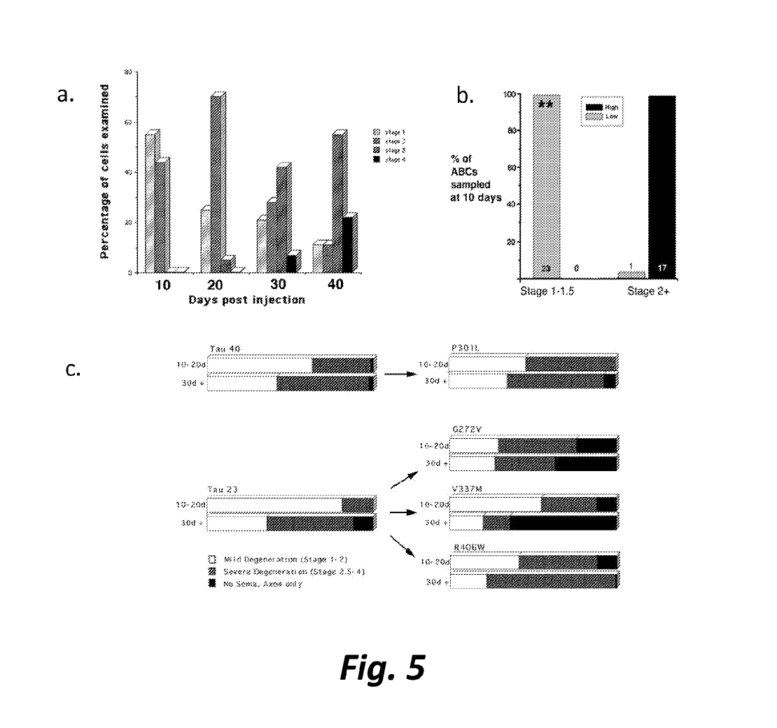

This Example illustrates that tau-induced degeneration is time and dose dependent and is accelerated by “tauopathy” mutations. Tau-induced degeneration in ABCs was used to demonstrate that this degeneration is progressive and that the rate of progression is increased with high levels of tau expression (defined in relation to endogenous tubulin levels). These data clearly show that the number of cells at later stages of degeneration increased with time following injection of the plasmid expressing tau (FIG. 5 a and b).

It was also demonstrated that the presence of tauopathy mutations (e.g., G272V, P301L, V337M, R406W) increases the proportion of high stage cells seen at both early (10-20 days of expression) and late (30 or more days of expression) times after plasmid injection relative to that seen with the expression of the parent WT isoform of tau (FIG. 5c).

### Example 4

This Example illustrates that the secretion of N terminal tau fragments is selective, restricted to live cells and is blocked by the presence of the amino acid sequence encoded by exon 2.

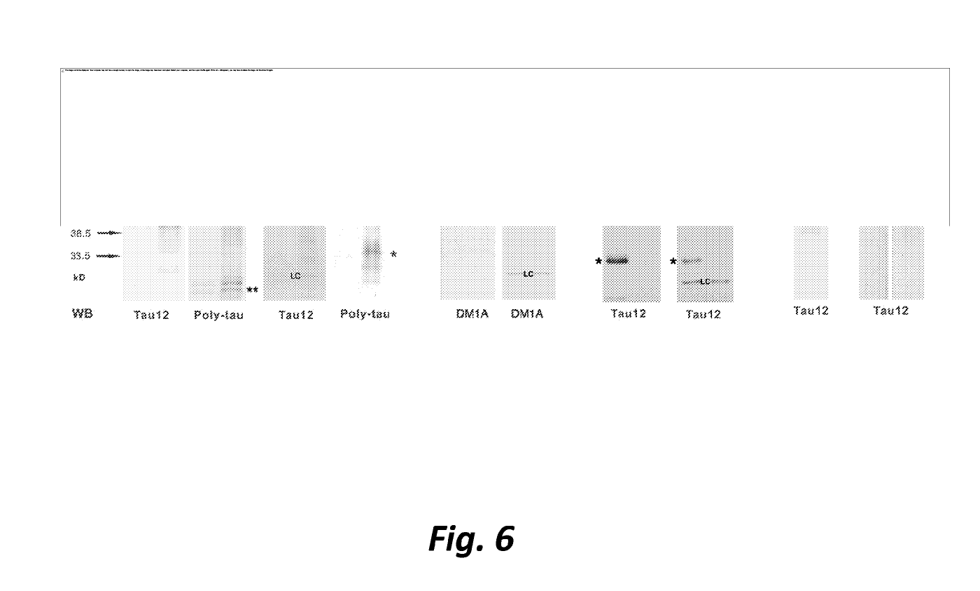

Western Blots (WB) of tau in cell lysates (left) and immunoprecipitated (IP) from the cell culture medium were performed using the mAb Tau12, which is specific to residues 12-18 on the tau N terminal. As shown in FIG. 6A, tau is secreted by healthy neurons and secretion of tau proteolytic cleavage fragments, including the tau N-terminal region, occurs with variable efficiency depending on the tau fragment and does not occur at all with some tau fragments. As shown in FIG. 6B, alpha tubulin is not secreted by the same calls, demonstrating that the secretion is specific to tau.

NB2A cells transiently transfected with plasmids encoding the N terminal half of tau (residues 1-255) either lacking (left lanes) or containing the exon 2/3 insert (right lanes) were tested for secretion of tau to the medium, using Tau12 for immunoprecipitation and Western Blotting (FIG. 6C). Note that nearly intact peptides of the 2/3− tau N terminal domain are secreted with relatively high efficiency, (*) while 2/3+ tau is retained in the lysate, illustrating the dependence of the secretion of N terminal tau on the absence of residues 45-102.

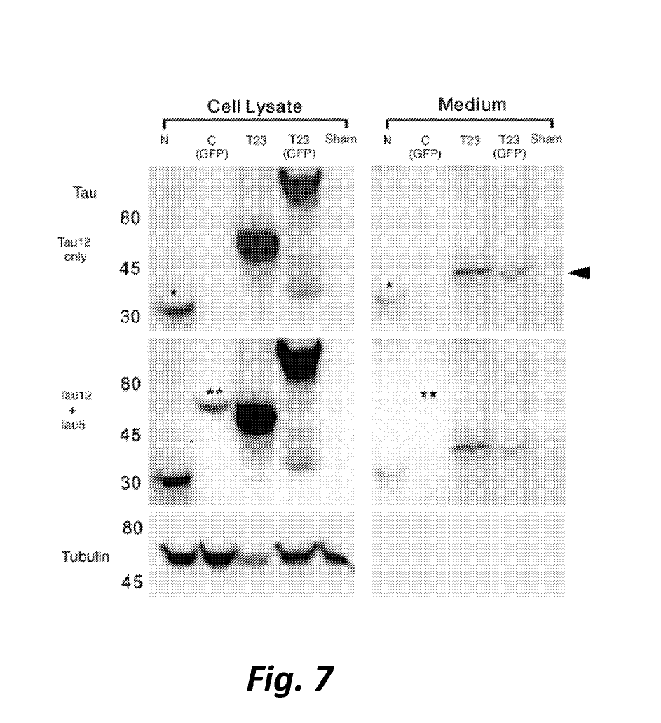

FIG. 7 also illustrates that the N-terminal mAb tau 12 (residues 9-18) recognizes secreted 1-255 and full length tau in concentrated culture medium conditioned by tau-expressing NB2A cells (FIG. 7—asterisk). Secreted full-length tau species shows some C-terminal cleavage (FIG. 7—caret). When the blot is re-probbed with an antibody specific for the Tau 5 epitope (specific for residues 210-235), it was observed that the C-terminal end (residues 211-441) is retained in the cell lysate (double asterisks) and the tau 5 epitope is absent from most of the secreted tau. Tubulin immunolabel was retained in the cell lysate, which indicates that tau secretion is not merely an artifact of non-specific release from degenerating NB2A cells (FIG. 7).

A comparison of the secretion of full length tau 2+ protein and tau 2− protein isoforms of nearly identical size (3RN1 and 4RN0, respectively) in the M1C cell line using medium concentration via microfiltration (Centricon) was performed to confirm the results derived from the above NB2A transient transfection experiments. (FIG. 6D) Note that the isoform lacking the exon 10 insert, but containing the exon 2 insert (3RN1) is secreted to a much lesser degree (about 20 times less) than is the exon 10+, exon 2− construct (4RN0). The use of medium concentration (rather than IP) shows clearly that the only variable associated with tau secretion is the absence/presence of an exon 2 or exon 2/3 sequence (FIG. 6D).

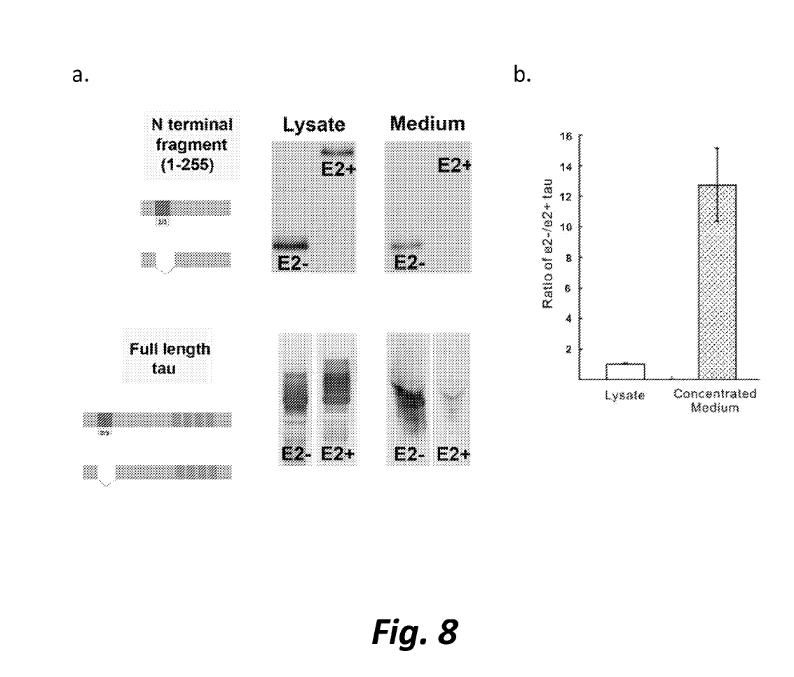

FIG. 8 clearly shows that in the presence of an N-terminal insert (E2), tau secretion is inhibited in both M1C and NB2A cells. Furthermore, this observation was found to be true for both IP and centrifuge-cencentrated media samples, cells with transient or induced tau expression and full length and N-terminal tau constructs. The data also clearly illustrate that tau without the E2 insert is secreted into the media 10-15 times more efficiently that tau comprising an E2 insert. (FIG. 8b).

### Example 5

This Example illustrates the mechanisms of tau secretion in lamprey ABCs and human neuroblastoma (M1C) cells. Expression of full length human tau isoforms (T23, T24, T40) in ABCs produces 2 tau secretion patterns referred to as diffuse and focal deposits. Diffuse Tau deposits are broadly distributed perisomatic deposits. Focal deposits are localized around degenerating dendrites near the point of onset of degenerations (POD). It is noteworthy that degeneration then proceeds along a distal to proximal gradient with time.

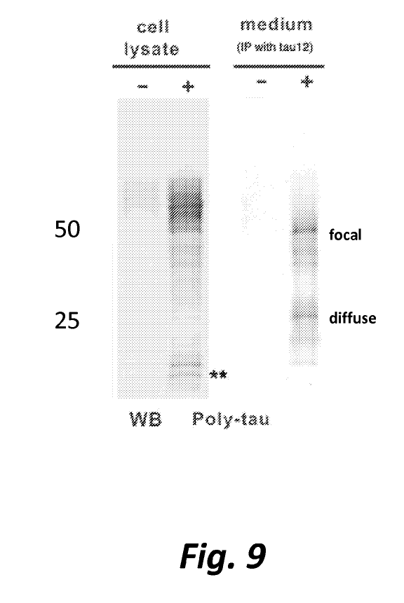

It has been determined that whether tau secretion occurs focally or in a diffuse pattern depends on the presence of the tau microtubule binding region (MTBR). The data clearly shows that diffuse (d) and focal (f) EC deposits have distinctive immunolabel patterns. (FIG. 9)

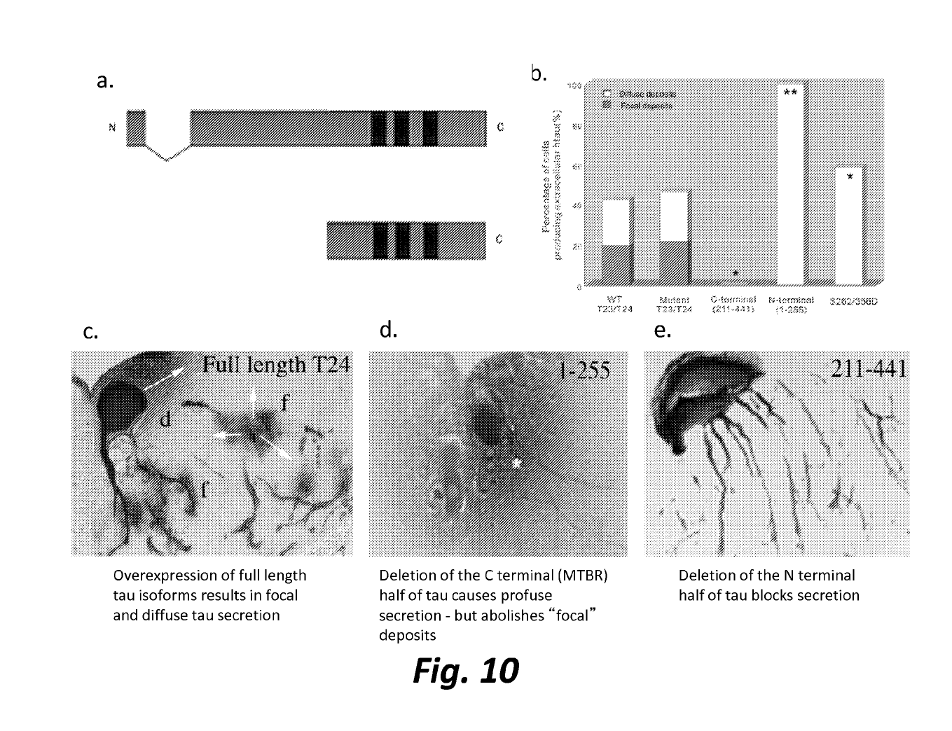

It has also been discovered that both diffuse and focal pathways of tau secretion from ABCs require the N terminal domain of tau (FIG. 10 a and b). Furthermore, overexpression of full length tau isoforms has been found to result in focal and diffuse tau secretion. Deletion of the C-terminal half of tau, which contains the MTBR, causes profuse secretion of tau, but abolishes focal deposits of the protein. In contrast, deletion of the N terminal half of tau has been found to block secretion of tau (FIG. 10 c-e).

### Example 6

This Example illustrates that the absence of the exon 2-3 insert in the tau amino terminal domain is highly correlated with tau secreted to the CSF from live neurons in the lamprey model.

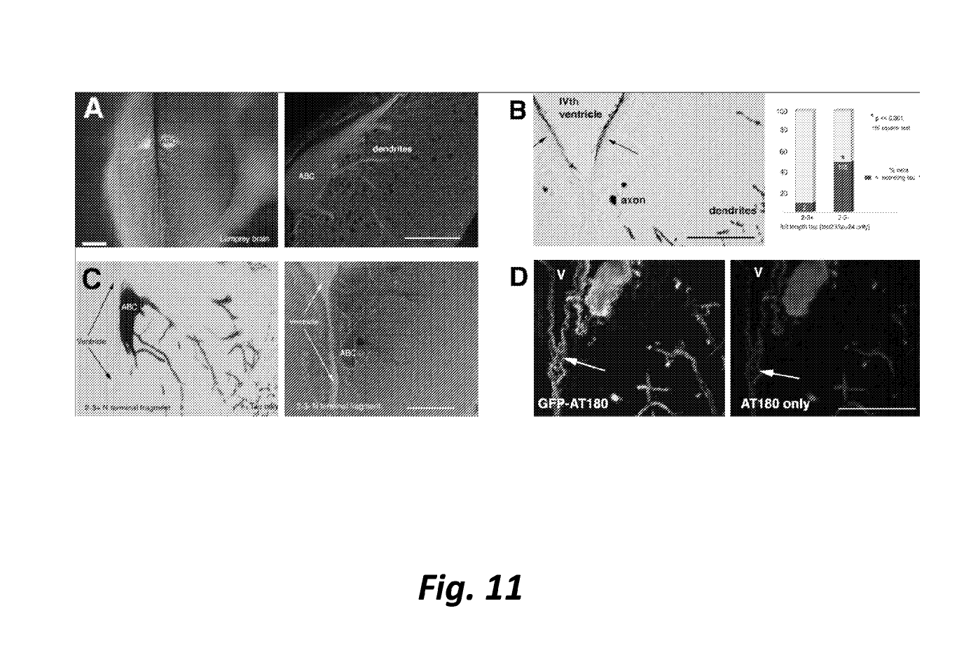

A transverse section of a lamprey brain was imaged by immunostaining with the N-terminal specific tau mAb taul2 (FIG. 11B). The data clearly shows that secreted tau reaches and crosses the IVth ventricle of the brain (arrows). Note that tau has been deposited along the ependymal layer lining the IVth ventricle at a point some distance caudal to the neurons secreting tau (FIG. 11B, left). Secretion is significantly correlated with the absence of the exon 2/3 insert in all constructs examined, which were scored on a per cell basis for secretion to the ECF (FIG. 11B, right). The difference is further illustrated in FIG. 11C (right side of Figure), in which the secretion of (1-255) N terminal tau fragments from ABCs is compared. Again, the exon 2/3+ fragment (left) is retained, whereas the exon 2/3− fragment is secreted with great efficiency (right). Secreted tau species that cross the IVth ventricle were observed to be immunolabeled with the phosphotau specific mAb AT180 (arrow, right), but with less efficiency than a marker of total secreted tau (i.e. polyclonal immunolabel of the N-terminal fusion GFP tag (left) (FIG. 11D). Note also that AT180+ tau is more retained in the ABC soma than is the GFP tag (left, soma profile).

A retrospective analysis of all ABCs that expressed full length tau isoforms at relatively high (St 2+) levels of expression after 10 days ppi was performed, and it was found that a similar pattern of expression held true in situ.

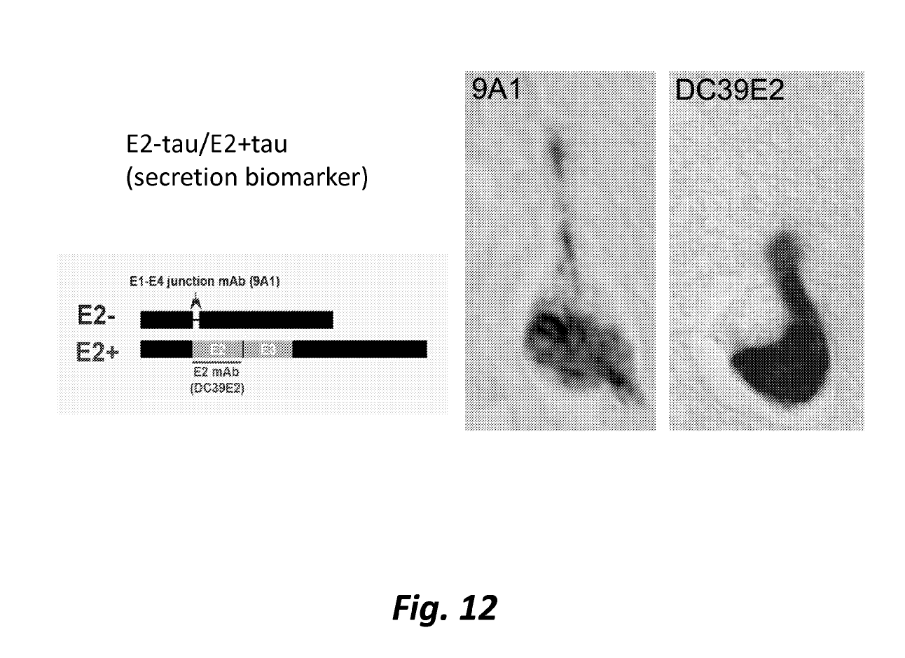

It is expected that tau secretion correlates with AD onset in human CSF and brain samples, and will be analyzed using commercially available monoclonal antibodies specifis for E2− (secreted) and E2+(retained) tau (FIG. 12).

### Example 7

This Example illustrates the expected results of a study comparing the sensitivity/specificity of the methods of the invention with conventional CSF assays. CSF samples from MCI (i.e. prodromal AD) and confirmed AD patients can be obtained from the Alzheimer's Brain Bank at Boston University Medical College (BUMCBB). CSF samples from normal, cognitively intact, age matched individuals can be used as negative controls, and will be expected to contain low levels of tau 2− protein (9A1+), tau 2+ protein (DC39E2+) and total tau (Tau12). CSF samples from patients with severe head trauma (traumatic brain injury or TBI) who are known to have suffered widespread neuronal loss typically exhibit high CSF-tau levels can be used as positive controls for neuronal death, and will be expected to show relatively high levels of 2+ tau, since tau release after TBI should be due to nonspecific leakage of tau from large numbers of ischemic, dying neurons. Samples from MCI and AD patients with history of stroke, multi-infarct dementia or other conditions associated with CNS ischemia can, therefore, be excluded from the study.

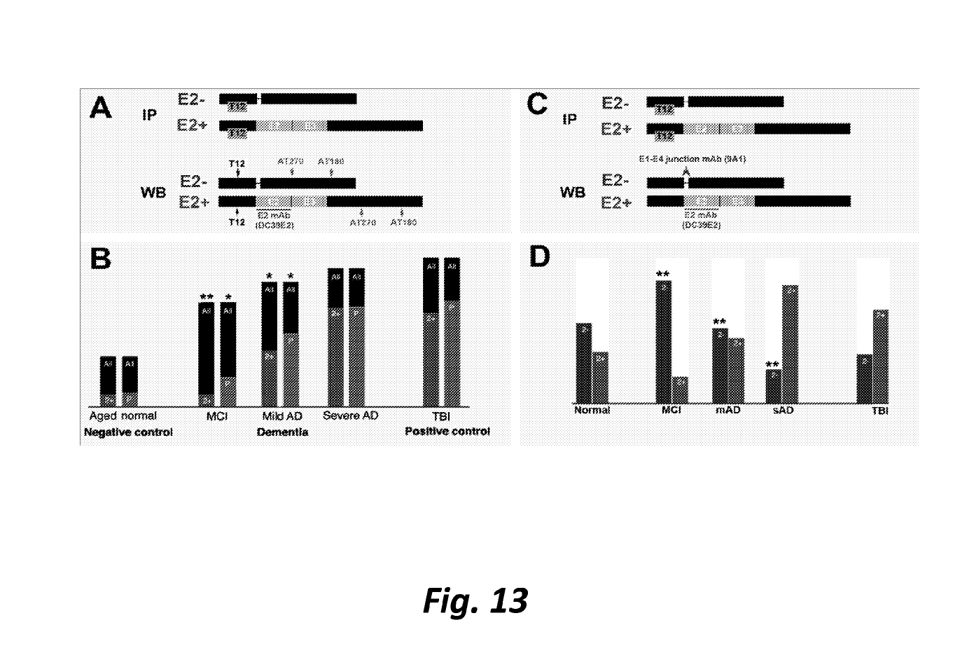

The diagnostic efficacy of the methods of the invention as compared to conventional methods can be assessed by comparing the tau 2+/2− ratio in CSF samples with AT180+ and AT270+ levels (the current standard measure of CSF tau phosphorylation (FIGS. 13A and 13B) and the results correlated with the cognitive state of the donor (routinely measured by the MMSE (Mini Mental Status Evaluation) at the time the CSF sample is taken at the BUMCBB) and/or with diagnosed status. 20-30 samples from each group can be analyzed. Aged normal individuals should have relatively low levels of non-MT-associated tau and low rates of tau turnover, leading to low levels of phosphorylation, cleavage and secretion of tau to the CSF. Since the rate of neuronal loss will be low, CSF levels of all tau species should be low.

For MCI, CSF samples should reflect the onset of degenerative changes leading to dementia but without widespread neuronal death, and thus should contain a highly significant increase in secreted tau 2− protein to the CSF (FIG. 13B,**). This may be accompanied by some neuronal death and possibly also by some secretion of Ptau/tau 2− protein, accounting for the observed increase in Ptau (FIG. 13B, *) that underlies the diagnostic value of current Ptau/total tau-based assays in MCI patients.

For AD, as dementia progresses from MCI through AD, tau released by neuron death (predominantly tau 2+ protein, since tau 2+ protein is both selectively retained and twice as common as tau 2− protein) should increasingly supplant secreted tau as the source of CSF tau, causing a sharp increase in the tau 2+/2− ratio (FIG. 13D,**). By contrast, AT180/270+ tau should increase directly but less dramatically as a function of cognitive decline (FIG. 5B), as tau is increasingly liberated to the CSF by neuronal death.

Since AT180+ is much less abundant in secreted tau than in retained tau in lamprey ABCs (FIG. 11D), the changes of tau 2+/2− ratio with diagnostic status and/or cognitive decline is expected to be more significant than that of the Ptau/Tau12 ratio (** vs *, respectively). For TBI, CSF samples from recent TBI patients should provide relatively high total levels of tau (varying with the severity of injury) with 2−/2+ tau levels close to the 63/37 ratio, since release will be almost completely due to the effects of acute neuronal death. Ptau/Tau12 ratios for TBI samples should also be augmented by kinase activity activated by high Ca++ levels in ischemic and necrotic neurons.

In sum, the tau 2+/2− ratio should be extremely sensitive to early changes associated with MCI, and the results should clearly indicate that a tau 2+/2− ratio-based diagnostic is more effective than current methodologies at diagnosing early stage tau-associated neurodegenerative disease (FIG. 13).

### Example 8

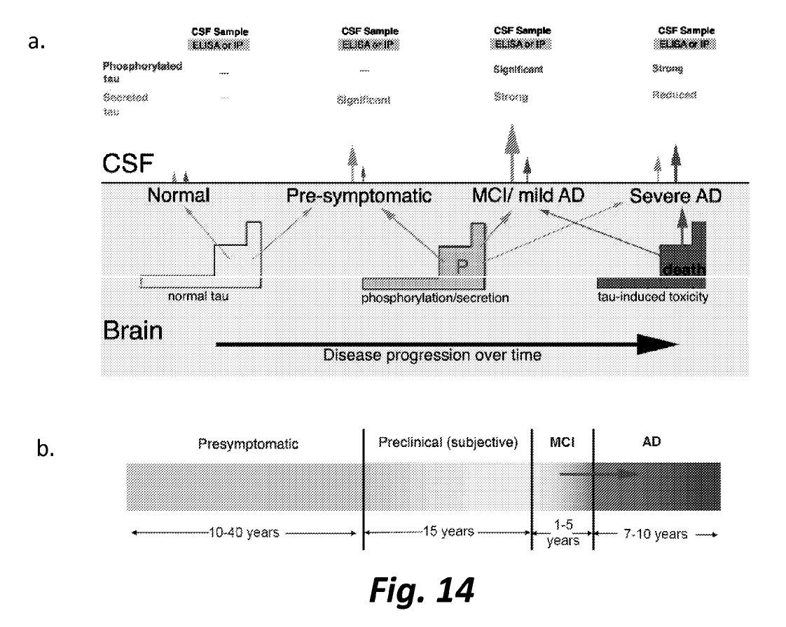

This Example illustrates the expected results that tau secretion can be used to develop a better tauopathy/AD diagnostic. FIG. 14a illustrates that the secreted tau protein may be the earliest biomarker of AD. In pre-symptomatic brains, a significant amount of secreted tau should be detected in the cerebrospinal fluid (CSF) when compared to normal brains. Furthermore, when CSF from patients with mild AD is probed for secreted tau, a stronger signal should be detected. Thus, the AD-associated protein tau should be secreted to the CSF before degeneration onset in subjects with AD. This invention should allow for the identification of AD patients before symptoms occur and is suitable for widespread screening of individuals at high risk for AD. Furthermore. this invention should be advantageous over current AD diagnostics which rely on neuron death markers in the CSF, which prevents the detection of AD before neuron loss occurs. In fact, current diagnostics use “death” biomarkers (e.g. phosphorylated tau) and can only identify AD after the onset of “mild cognitive impairment” (MCI). By this point, irreversible CNS damage may have occurred in the subject (FIG. 14b). Thus, identifying subjects with AD before irreversible changes occur may: 1. make treatments more effective, 2. identify pre-symptomatic AD patients for inclusion in current drug trials, and 3. identify “curable” AD patients for treatment in the future.

Thus, it is expected that the use of an identifiable AD biomarker (E2-tau) secreted from neurons before they degenerate will help improve diagnostics for AD. Furthermore, if secreted tau precedes “death” markers such as P-tau, E2-tau should be enriched in CSF samples from MCI/early AD patients.

### Example 9

This Example illustrates that tau is secreted in exosomes. Exosomes are a newly discovered, “unconventional” route of protein secretion. Because tau is not a classical secreted protein, prior to the instant invention, tau secretion had not been studied as a source of CSF tau. Interestingly, related proteins (e.g., tubulin, actin and associated proteins) that should not be secreted can, in fact, be secreted by this pathway. Other “non-secreted” disease associated proteins (e.g., amyloid beta protein (AD) and alpha synuclein (Parkinson's Disease)) are secreted by exosomes.

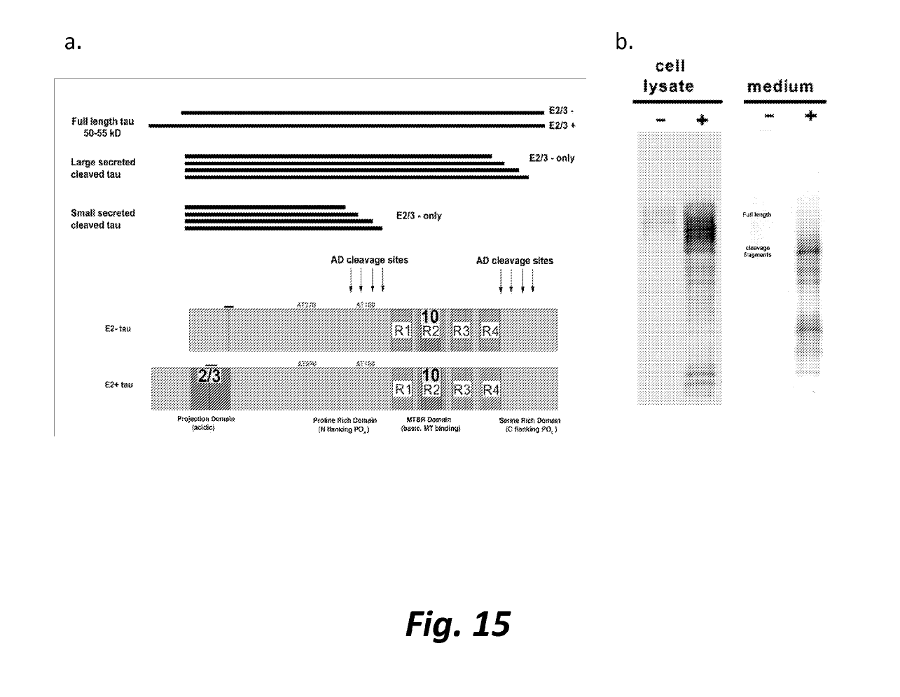

This Example further illustrates that tau is concentrated in the “exosome fraction” of cell culture medium and that secreted tau resembles CSF tau species (cleavage fragments) associated with AD. Furthermore, CSF tau in AD is cleaved by disease activating enzymes (caspases and calpains) that degrade proteins at specific sites; and the immunoblot depicted in FIG. 15 clearly illustrates that exosome secreted tau occurs primarily in 2 fragment sizes that resemble the caspase and calpain fragments observed in AD.

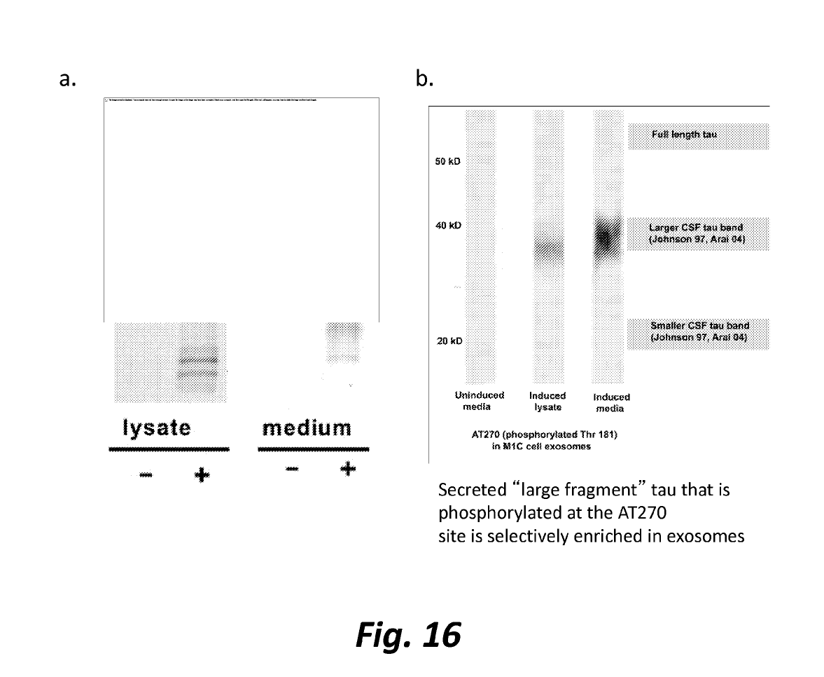

Figure P further illustrates that secreted tau associated with exosomes resembles CSF tau from early AD patients. Furthermore, these data show that the secreted “large fragment” tau that is phosphorylated at the AT270 site is selectively enriched in exosomes (FIG. 16).

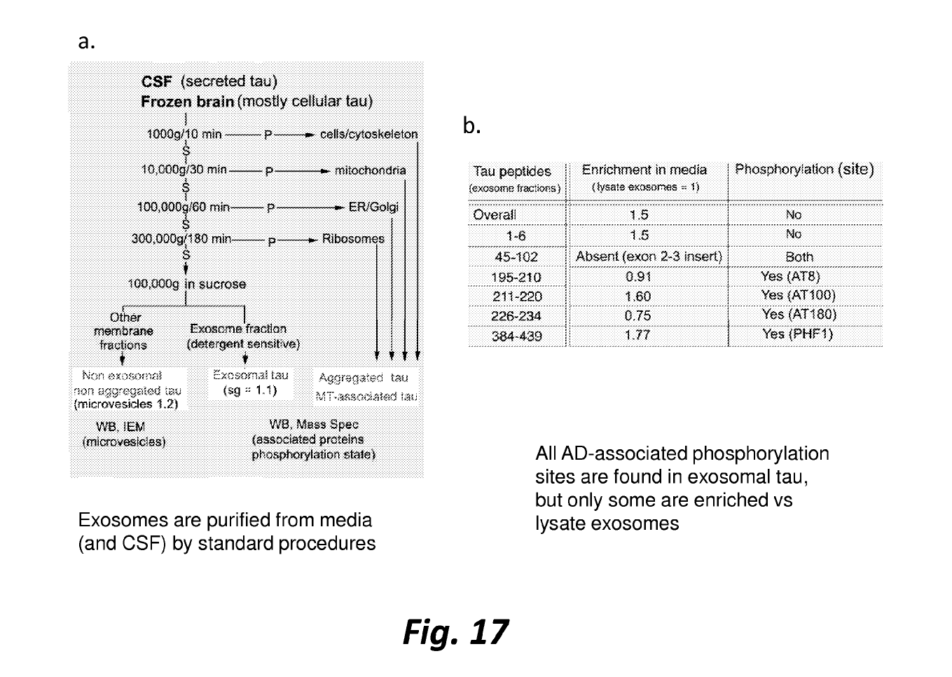

FIG. 17 schematically depicts the purification of microvesicle secreted tau and the purification of exosomal secreted tau from media and CSF

### Example 10

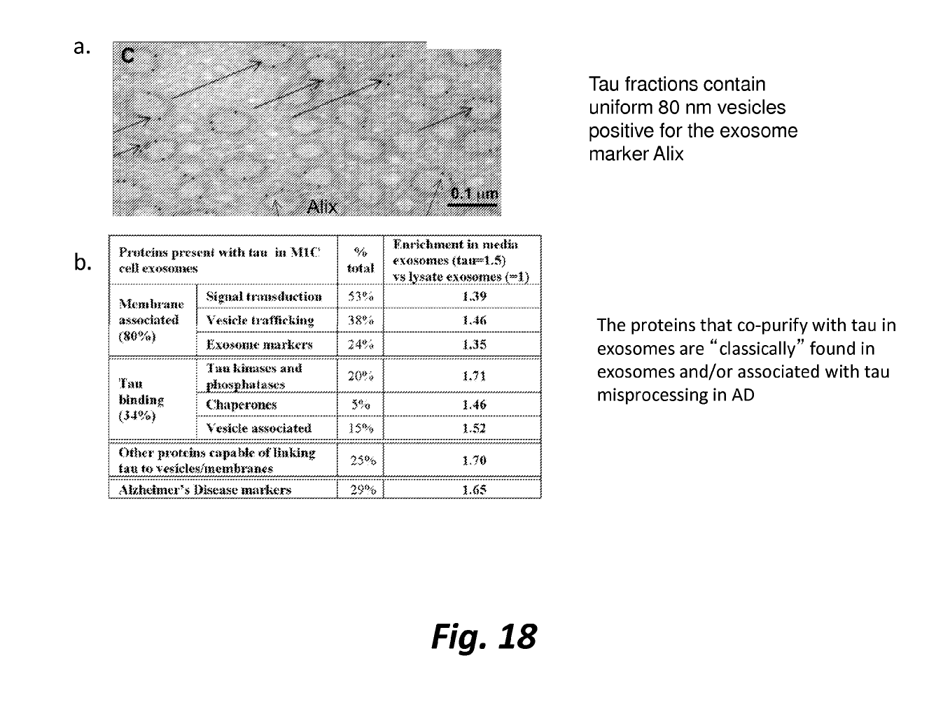

This Example illustrates that secreted tau is associated with morphologically typical exosomes and with exosome and AD-associated proteins. FIG. 18 clearly shows that tau fractions contain uniform 80 nm vesicles positive for the exosome marker Alix.

A variety of proteins co-purified in media exosome fractions with tau, of which 72 were specifically identified based on sequence databases⁵⁰. Most of these were membrane associated proteins with signal transduction functions, including several specific markers of exosomes (e.g. Alix—FIG. 18a). Almost all of the proteins (with the notable exception of tau) are members of families that have been identified in exosome preparations from various tissues and bodily fluids (neuroblastoma, urine, blood, cerebrospinal fluid) in earlier studies. Of these, 21 proteins which were both a) consistently seen in M1C media exosome fractions in significant amounts (top quartile peak height) and b) enriched in media exosomes relative to their concentration in exosome preparations from M1C lysates. These were characterized further for clues to the mechanism responsible for tau secretion and possible links to tau-related disease (FIG. 18). Of these, 34% (directly and/or indirectly) are known to bind tau and might therefore participate in its inclusion in exosomes and secretion, and another 25% (lipidating and glycosylating enzymes, or containing known tau-binding motifs) could link tau to membrane associated elements that are known to be released in exosomes. Furthermore, the proteins that co-purify with tau in exosomes are “classically” found in exosomes and/or associated with tau misprocessing in AD.

In addition, this Example further illustrates that tau secretion in lamprey occurs via vesicles containing fyn, an exosomal protein associated with AD. It was observed that the tyrosine kinase fyn, which selectively phosphrorylates tau in AD, colocalizes at the vesicles that are undergoing exocytosis from ABC dendrites.

1. A method for identifying a subject having, or at risk of developing, a tau-associated neurodegenerative disease comprising obtaining a biological sample from the subject and making a determination selected from the group consisting of:
a) determining the amount of tau 2+ protein in the biological sample relative to a suitable control, wherein a decreased amount of tau 2+ protein relative to the control identifies the subject as having, or being at risk of developing, a tau-associated neurodegenerative disease;
b) determining the amount of tau 2− protein in the biological sample relative to a suitable control, wherein a increased amount of tau 2− protein relative to the control identifies the subject as having, or being at risk of developing, a tau-associated neurodegenerative disease;
c) determining the tau 2+/tau 2− ratio in the biological sample relative to a suitable control, wherein a decreased tau 2+/tau 2− ratio relative to the control identifies the subject as having, or being at risk of developing, a tau-associated neurodegenerative disease; and
d) determining the amount of secreted vesicular tau protein in the biological sample relative to a suitable control, wherein an increased amount of secreted vesicular tau protein relative to the control identifies the subject as having, or being at risk of developing, a tau-associated neurodegenerative disease.

2-4. (canceled)

5. The method of claim 1, wherein determining the increased amount of secreted vesicular tau protein comprises determining an increased amount of tau 2− protein.

6. The method of claim 1, wherein determining the increased amount of secreted vesicular tau protein comprises determining a decreased amount of tau 2+ protein.

7. The method of claim 1, wherein determining the increased amount of secreted vesicular tau protein comprises determining a decreased tau 2+/tau 2− ratio.

8. The method of 1 wherein the subject is a mammal.

9. The method of claim 8, wherein the subject is a human.

10. The method of claim 1, wherein the subject has no clinically discernible cognitive impairment.

11. The method of claim 1, wherein the a tau-associated neurodegenerative disease is selected from the group consisting of Alzheimer's Disease, corticobasal degeneration, Pick's Disease, progressive supernuclear palsy, granulovacuolar disease, frontotemporal dementia, Lewy Body disease, Creutzfeld-Jacob Disease (CJD), variant Creutzfeld-Jacob Disease, and new variant Creutzfeld-Jacob Disease.

12. A method for identifying a tau abnormality in a biological sample, comprising making a determination selected from the group consisting of:
a) determining the amount of tau 2+ protein in the biological sample relative to a suitable control, wherein a decreased amount of tau 2+ protein relative to the control identifies a tau abnormality;
b) determining the amount of tau 2− protein in the biological sample relative to a suitable control, wherein a increased amount of tau 2− protein relative to the control identifies a tau abnormality; and
c) determining the tau 2+/tau 2− ratio in the biological sample relative to a suitable control, wherein a decreased tau 2+/tau 2− ratio relative to the control identifies a tau abnormality.

13-14. (canceled)

15. The method of claim 1 or 12, wherein the biological sample is an extracellular fluid.

16. The method of claim 15, wherein the extracellular fluid is cerebrospinal fluid.

17. The method of claim 15, wherein the extracellular fluid is blood, serum or plasma.

18. The method of claim 1 or 12, wherein the amount of tau 2+ protein, tau 2− protein or the tau 2+/tau 2− ratio is determined by Western Blotting, ELISA, dot-blotting, high performance liquid chromatography (HPLC) or mass spectrometry.

19. The method of claim 1 or 12, wherein the tau 2+ protein is detected using an antibody that specifically binds to the amino acid sequence set forth in SEQ ID No.7.

20. The method of claim 1 or 12, wherein the tau 2+ protein is detected using an antibody that specifically binds to the amino acid sequence set forth in SEQ ID No.8.

21. The method of claim 1 or 12, wherein the tau 2− protein is detected using an antibody that specifically binds to the Exon 1/Exon 4 junction region of human tau.

22. The method of claim 1 or 12, wherein the tau 2− protein is detected using an antibody that specifically binds to the Exon 1/Exon 3 junction region of human tau.

23. A kit for identifying a tau abnormality in a biological sample, the kit comprising:
a) means for determining the amount of tau 2+ protein, tau 2− protein or the tau 2+/tau 2− ratio; and
b) instructions for use of the kit to identify a tau abnormality in the sample.

24. The kit of claim 23, further comprising a means for obtaining the biological sample.

25. The kit of claim 23 or 24, further comprising a suitable control sample.


SEQUENCE LISTING (8 sequences)

SEQ ID NO: 1 - protein, 758 aa, Homo sapiens
  misc_feature: Human tau isoform 1
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKHPTPGSSDPLIQPSSPAVCPEPPSSPKHVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 2 - protein, 441 aa, Homo sapiens
  misc_feature: Human tau isoform 2
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 3 - protein, 383 aa, Homo sapiens
  misc_feature: Human tau isoform 3
MAEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 4 - protein, 352 aa, Homo sapiens
  misc_feature: Human tau isoform 4
MAEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 5 - protein, 412 aa, Homo sapiens
  misc_feature: Human tau isoform 5
MAEPRQEFEVMEDHAGTYGLGDRKDQGGYTMHQDQEGDTDAGLKESPLQTPTEDGSEEPGSETSDAKSTPTAEAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 6 - protein, 776 aa, Homo sapiens
  misc_feature: Human tau isoform 6
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKHPTPGSSDPLIQPSSPAVCPEPPSSPKHVSSVTSRTGSSGAKEMKLKGADGKTKIATPRGAAPPGQKGQANATRIPAKTPPAPKTPPSSATKQVQRRPPPAGPRSERGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 7 - protein, 30 aa, Homo sapiens
  misc_feature: Amino acid sequence encoded by human Exon 2
ESPLQTPTEDGSEEPGSETSDAKSTPTAED

SEQ ID NO: 8 - protein, 29 aa, Homo sapiens
  misc_feature: Amino acid sequence encoded by human Exon 3
DVTAPLVDEGAPGKQAAAQPHTEIPEGTT